(19) United States
(12) Patent Application Publication
(10) Pub. No.: US 2008/0077025 A1
(43) Pub. Date: Mar. 27, 2008

(54) METHODS FOR DETERMINING HOW TO TREAT CONGESTIVE HEART FAILURE

(76) Inventors: Leticia Delgado-Herrera, Lake Forest, IL (US); Robert J. Padley, Lake Villa, IL (US); Jeffrey E. Salon, New Albany, OH (US); Roopal B. Thakkar, Grayslake, IL (US); Bidan Huang, Vernon Hills, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/456,741

(22) Filed: Jul. 11, 2006

Related U.S. Application Data
(60) Provisional application No. 60/779,266, filed on Mar. 3, 2006.
(60) Provisional application No. 60/735,613, filed on Nov. 12, 2005.
(60) Provisional application No. 60/698,173, filed on Jul. 11, 2005.

(51) Int. Cl. A61B 5/02; G06Q 50/00

(52) U.S. Cl. 600/508; 705/14; 705/2

(57) ABSTRACT

The present invention relates to methods for determining how to treat congestive heart failure.

The present application claims priority to U.S. Provisional Application Ser. No. 60/779,266, filed Mar. 3, 2006; U.S. Provisional Application Ser. No. 60/735,613, filed Nov. 12, 2005; and U.S. Provisional Application Ser. No. 60/698,173, filed Jul. 11, 2005, hereby incorporated their entirety by reference.

### FIELD OF THE INVENTION

The present invention relates to methods of predicting the likelihood of an improved clinical outcome in a patient diagnosed with congestive heart failure, methods of determining whether or not to begin a course of monotherapy or combination therapy in a patient diagnosed with congestive heart failure, methods for determining whether to continue or discontinue a course of monotherapy or combination therapy in a patient diagnosed with congestive heart failure and methods of optimizing a treatment regimen in a patient diagnosed with congestive heart failure.

### BACKGROUND OF THE INVENTION

Congestive heart failure (CHF) is a condition in which weakened heart function exists together with a build-up of body fluid. Congestive heart failure often occurs when cardiac output is insufficient to meet the metabolic demands of the body, or when the heart cannot meet the demands of operating at increased levels of filling/diastolic pressure. Treating patients diagnosed with CHF involves not only support of the weakened heart function but also treatment to counteract the build up of the body fluid.

Congestive heart failure may be caused by many forms of heart disease. Common causes of CHF include: narrowing of the arteries supplying blood to the heart muscle (coronary heart disease); prior heart attack (myocardial infarction) resulting in scar tissue large enough to interfere with normal function of the heart; high blood pressure; heart valve disease due to past rheumatic fever or an abnormality present at birth; primary disease of the heart muscle itself (cardiomyopathy); defects in the heart present at birth (congenital heart disease) and infection of the heart valves and/or muscle itself (endocarditis and/or myocarditis). Each of these disease processes can lead to CHF by reducing the strength of the heart muscle contraction, by limiting the ability of the heart's pumping chambers to fill with blood due to mechanical problems or impaired diastolic relaxation, or by filling the heart's chambers with too much blood.

CHA includes both acute and chronic presentations. Typically, in both acute and chronic presentations, advanced CHF may involve patients who are decompensated. Patients presenting with acutely decompensated CHF can have an acute injury to the heart, such as a myocardial infarction, mitral regurgitation or ventricular septal rupture. Typically, the injury compromises myocardial performance (for example, a myocardial infarction) or valvular/chamber integrity (for example, mitral regurgitation or ventricular septal rupture). Such injuries result in an acute rise in the left ventricular (LV) filling pressures. The rise in the LV filling pressures results in pulmonary edema and dyspnea. The treatment of patients with acutely decompensated CAF focuses on temporarily stabilizing the patient and/or morbidities associated with the patients overall critical condition. This typically is accomplished by treating the immediate and apparent conditions associated with heart failure. In addition, the heart's function is supported by treatments to reduce LV filling pressures and to improve cardiac performance.

Patients with chronic decompensated CHF often have symptoms of volume overload and/or low cardiac output. These symptoms are associated with chronic LV systolic dysfunction.

Current standard of care protocols, involve treating CAF patients with agents that decrease volume (such as diuretics), decrease the work the heart needs to do by reducing after load (such as vasodilators), and by increasing the contractility of the heart (such as inotropic drugs). However, it is unclear which patients are best served by the above therapies.

The present invention aids in treating these problems by providing that certain blood pressure measurements in patients diagnosed with congestive heart failure can be used to predict whether these patients have an increased likelihood of an improved clinical outcome, to determine whether or not to begin a course of monotherapy or combination therapy in these patients, to determine whether to continue or discontinue a course of monotherapy or combination therapy in these patients and to optimize an existing treatment regimen in these patients.

### SUMMARY OF THE INVENTION

In one embodiment, the present invention relates to a method of predicting an improved clinical outcome in a patient diagnosed with congestive heart failure. The method involves the following steps:

(a) obtaining a systolic blood pressure, diastolic blood pressure or mean arterial pressure from a patient;

(b) determining whether the systolic blood pressure, diastolic blood pressure or mean arterial pressure of said patient fits at least one predetermined criterion showing a likelihood of an improved clinical outcome; and

(c) predicting said improved clinical outcome based upon the determination in step (b).

The predetermined criteria used in the above method to show a likelihood of improved clinical outcome is a systolic blood pressure about equal to or greater than 100 mmHg, a diastolic blood pressure about equal to or greater than 60 mmHg or a mean arterial pressure about equal to or greater than 75 mmHg.

For example, in the above method, when the patient's systolic blood pressure is about equal to or greater than 100 mmHg this indicates that the patient has an increased likelihood of an improved clinical outcome. In contrast, when the patient's systolic blood pressure is less than about 100 mmHg, this indicates that the patient has a decreased likelihood of an improved clinical outcome.

The above-described method can be used in any congestive heart failure patient, including, but not limited to, a patient that is suffering from acutely decompensated congestive heart failure.

In a second embodiment, the present invention relates to a method for determining whether or not to begin a course of monotherapy with an isotropic drug or a vasodilator in a patient diagnosed with congestive heart failure. This method involves the following steps:

(a) obtaining a systolic blood pressure, diastolic blood pressure or mean arterial pressure from a patient;

(b) analyzing the systolic blood pressure, diastolic blood pressure or mean arterial pressure of said patient to determine whether said blood pressure, diastolic blood pressure or mean diastolic blood pressure fits at least one predetermined criterion; and

(c) determining whether to begin a course of monotherapy in the patient based upon the analysis in step (b).

The predetermined criteria used in the above method is a systolic blood pressure less than, equal to or greater than about 100 mmHg, a diastolic blood pressure less than, equal to or greater than about 60 mmHg or a mean arterial pressure less than, equal to or greater than about 75 mmHg.

For example, in the above method, if a patient is determined to have a systolic blood pressure less than about 100 mmHg in step (b), then in step (c), a determination or decision would be made not to begin a course of monotherapy with an inotropic drug or a vasodilator in said patient. In contrast, if a patient is determined to have a systolic blood press that is equal to or greater than about 100 mmHg in step (b), then in step (c), a determination or decision would be made to begin a course of monotherapy with an inotropic drug or vasodilator.

If in the above method, a determination or decision is made in step (c) to begin a course of monotherapy, then the method may further involve the step of administering an effective amount of an inotropic drug or a vasodilator to the patient. The inotropic drug or vasodilator can be administered to the patient orally, buccally, intravenously, intramuscularly, subcutaneously or by inhalation.

The above-described method can be used in any congestive heart failure patient, including, but not limited to, a patient that is suffering from acutely decompensated congestive heart failure.

In alternative embodiment, the present invention relates to a method for determining whether or not to begin a course of combination therapy with at least one inotropic drug, at least one vasodilator or combinations thereof in a patient diagnosed with congestive heart failure. The method involves the following steps:

(a) obtaining a systolic blood pressure, diastolic blood pressure or mean arterial pressure from a patient;

(b) analyzing the systolic blood pressure, diastolic blood pressure or mean arterial pressure of said patient to determine whether said blood pressure, diastolic blood pressure or mean diastolic blood pressure fits at least one predetermined criterion; and

(c) determining whether to begin a course of combination therapy with at least one inotropic drug, at least one vasodilator or combinations thereof in the patient based upon the analysis in step (b).

The predetermined criteria used in the above method is a systolic blood pressure less than, equal to or greater than about 100 mmHg, a diastolic blood pressure less than, equal to or greater than about 60 mmHg or a mean arterial pressure less than, equal to or greater than about 75 mmHg.

For example, in the above method, if a patient is determined to have a systolic blood pressure less than about 100 mmHg in step (b), then in step (c), a determination or decision would be made not to begin a course of combination therapy with at least one inotropic drug, at least one vasodilator or combinations thereof in said patient. In contrast, if a patient is determined to have a systolic blood press that is about equal to or greater than about 100 mmHg in step (b), then in step (c), a determination or decision would be made to begin a course of combination therapy with at least one inotropic drug, at least one vasodilator or combinations thereof.

If in the above method, a determination or decision is made in step (c) to begin a course of combination therapy, then the method may further involve the step of administering an effective amount of at least one inotropic drug, at least one vasodilator or combinations thereof to the patient. The at least one inotropic drug, at least one vasodilator or combinations thereof can be administered to the patient orally, buccally, intravenously, intramuscularly, subcutaneously or by inhalation.

The above described method can be used in any congestive heart failure patient, including, but not limited to, a patient that is suffering from acutely decompensated congestive heart failure.

In a third embodiment, the present invention relates to a method for determining whether to continue or discontinue a course of monotherapy in a patient diagnosed with congestive heart failure wherein said patient is currently being treated with an inotropic drug or a vasodilator. The method involves the following steps:

(a) obtaining a systolic blood pressure, diastolic blood pressure or mean arterial pressure from a patient;

(b) analyzing the systolic blood pressure, diastolic blood pressure or mean arterial pressure of said patient to determine whether said systolic blood pressure, diastolic blood pressure or mean diastolic blood pressure fits at least one predetermined criterion; and

(c) determining whether to continue or discontinue the course of monotherapy with the inotropic drug or vasodilator in said patient based upon the analysis in step (b).

The predetermined criteria used in the above method is a systolic blood pressure less than, equal to or greater than about 100 mmHg, a diastolic blood pressure less than, equal to or greater than about 60 mmHg or a mean arterial pressure less than, equal to or greater than about 75 mmHg.

For example, in the above method, if a patient is determined to have a systolic blood pressure less than about 100 mmHg in step (b), a determination or decision will be made in step (c) not to continue the course of monotherapy with an inotropic drug or vasodilator in the patient. In contrast, if a patient is determined to have a systolic blood pressure about equal to or greater than about 100 mmHg in step (b), a determination or decision will be made in step (c) to continue the course of monotherapy with an inotropic drug or, vasodilator in the patient.

The above described method can be used in any congestive heart failure patient, including, but not limited to, a patient that is suffering from acutely decompensated congestive heart failure.

In an alternative embodiment, the present invention relates to a method for determining whether to continue or discontinue a course of combination therapy in a patient diagnosed with congestive heart failure wherein said patient is currently being treated with a combination of at least one inotropic drug, at least one vasodilator or combinations thereof. The method involves the steps of:

(a) obtaining a systolic blood pressure, diastolic blood pressure or mean arterial pressure from a patient;

(b) analyzing the systolic blood pressure, diastolic blood pressure or, mean arterial pressure of said patient to determine whether said systolic blood pressure, diastolic blood pressure or mean diastolic blood pressure fits at least one predetermined criterion; and

(c) determining whether to continue or discontinue the course of combination therapy with the at least one inotropic drug, at least one vasodilator or combinations thereof in said patient based upon the analysis in step (b).

The predetermined criteria used in the above method is a systolic blood pressure less than, equal to or greater than about 100 mmHg, a diastolic blood pressure less than, equal to or greater than about 60 mmHg or a mean arterial pressure less than, equal to or greater than about 75 mmHg.

For example, in the above method, if a patient is determined to have a systolic blood pressure less than about 100 mmHg in step (b), a determination or decision will be made in step (c) not to continue the course of combination therapy with at least one inotropic drug, at least one vasodilator or combinations thereof in the patient. In contrast, if a patient is determined to have a systolic blood pressure equal to or greater than about 100 mmHg in step (b), a determination or decision will be made in step (c) to continue the course of combination therapy with at least one inotropic drug, at least one vasodilator or combinations thereof in the patient.

The above described method can be used in any congestive heart failure patient, including, but not limited to, a patient that is suffering from acutely decompensated congestive heart failure.

In a fourth embodiment, the present invention relates to a method of optimizing an existing drug treatment regimen in a patient diagnosed with congestive heart failure and receiving at least one inotropic drug, at least one vasodilator, at least one beta-blocker or combinations thereof. The method involves the following steps:

(a) obtaining a systolic blood pressure, diastolic blood pressure or mean arterial pressure from a patient;

(b) analyzing the systolic blood pressure, diastolic blood pressure or mean arterial pressure of said patient to determine whether said systolic blood pressure, diastolic blood pressure or mean diastolic blood pressure fits at least one predetermined criterion; and

(c) optimizing the treatment regimen in the patient based upon the analysis in step (b).

The predetermined criteria used in the above method is a systolic blood pressure less than, equal to or greater than about 100 mmHg, a diastolic blood pressure less than, equal to or greater than about 60 mmHg or a mean arterial pressure less than, equal to or greater than about 75 mmHg.

For example, in the above method, if a patient is determined to have a systolic blood pressure less than about 100 mmHg in step (b), a determination or decision will be made in step (c) to optimize the treatment regimen by discontinuing the treatment of the patient with at least one inotropic drug, at least one vasodilator, at least one beta-blocker or combinations thereof. Alternatively, if a patient is determined to have a systolic blood pressure equal to or greater than about 100 mmHg in step (b), a determination or decision can be made in step (c) to optimize the treatment regimen by substituting an effective amount of at least one different inotropic drug, at least one different vasodilator, at least one different beta-blocker or different combinations thereof that is not currently being used in the existing treatment regimen for at least one inotropic drug, at least one vasodilator, at least one beta-blocker or combinations thereof that is currently being used in the existing treatment regimen. Alternatively, if a patient is determined to have a systolic blood pressure equal to or greater than about 100 mmHg in step (b), a determination or decision can be made in step (c) to optimize the treatment regimen by adding an effective amount of at least one inotropic drug, at least one vasodilator, at least one beta-blocker or combinations thereof that is not currently being used in the existing treatment regimen.

The above described method can be used in any congestive heart failure patient, including, but not limited to, a patient that is suffering from acutely decompensated congestive heart failure.

In another embodiment, the invention comprises a pharmaceutical drug product comprising: (a) a pharmaceutical drug for the treatment of congestive heart failure in a pharmaceutically-acceptable form for administration to a human, and (b) printed media or labeling accompanying the pharmaceutical drug, wherein said printed media or labeling discloses at least one of the methods described herein. Preferably, the pharmaceutical drug product is levosimendan.

In another embodiment, the invention comprises a business method comprising, providing a drug for the treatment of congestive heart failure in a pharmaceutically-acceptable form for administration to a human, and encouraging a decision maker to refer to printed or electronic media disclosing at least one of the methods described herein. Preferably, the pharmaceutical drug product is levosimendan.

In yet another embodiment, the invention comprises a method of promoting the sale or use of a drug for the treatment of congestive heart failure, the method comprising the steps of providing the drug in a pharmaceutically-acceptable form for administration to a human, and providing a customer with printed, graphic, or electronic media disclosing at least one of the methods described herein. Preferably, the pharmaceutical drug product is levosimendan.

### BRIEF DESCRIPTION OF THE FIGURES

FIG. 1A shows that in the REVIVE II study, patients treated with levosimendan experienced statistically significant decreases in BNP (without nesiritide (which is BNP)) at 24 hours and 5 days compared to patients treated with placebo and on a background of standard-of-care therapy. FIG. 1B shows the decrease in BNP from the REVIVE II study for patients who received nesiritide and who were also treated with levosimendan or placebo.

FIG. 2 shows comparison data in clinical potency (which is defined by the percentage of patients improved minus the percentage of patients worsened) for Patient Global Assessment in the REVIVE II study.

FIG. 3 shows the changes in the effect of clinical potency (which is defined by the percentage of patients improved minus the percentage of patients worsened) in patients treated with levosimendan compared to placebo in their dyspnea assessment.

FIG. 4 shows the renal function effects in patients treated with levosimendan compared to placebo. The top graph shows creatinine in mg/dL. The bottom graph shows blood urea nitrogen (BUN) in mg/dL.

FIG. 5 shows all cause mortality in the REVIVE II study of patients treated with levosimendan and placebo at 5, 14, 31 and 90 days.

FIG. 6 shows the primary endpoint analysis that excluded patients having a systolic blood pressure (SBP) less than 100 mmHg.

### DETAILED DESCRIPTION OF THE INVENTION

### Definitions

As used in this specification and the appended claims, the singular forms “a,” “an” and “the” include plural references unless the context clearly dictates otherwise.

In describing and claiming the present invention, the following terminology will be used in accordance with the definitions set out below.

The terms “administer”, “administering”, “administered” or “administration” refer to any manner of providing a drug (such as, but not limited to, at least one inotropic drug, at least one vasodilator, at least one beta-blocker or combinations thereof) to a subject or patient. Routes of administration can be accomplished through any means known by those skilled in the art. Such means include, but are not limited to, oral, buccal, intravenous, subcutaneous, intramuscular, by inhalation and the like.

As used herein, the term “beta-blocker” refers to a beta-adrenoreceptor drug that works by blocking the action of noradrenaline at receptors in the heart and circulatory system. Beta-blockers are used to lower high blood pressure, relieve angina, correct arrhythmias, prevent migraine headaches, reduce physical symptoms associated with anxiety and to relieve the symptoms associated with hyperthyroidism. Examples of beta-blockers include, but are not limited to, labetalol, carvedilol, atenolol, esmolol, esmolol hydrochloride, metoprolol, metoprolol succinate, metoprolol tartrate, bisoprolol fumarate, bisoprolol, propranolol or propranolol hydrochloride.

As used herein, the term “cyanosis” refers to a dark bluish or purplish coloration of the skin and mucous membrane due to deficient oxygenation of the blood. Methods for determining cyanosis are well known to those skilled in the art.

As used herein, the term “diaphoresis” refers to perspiration, especially profuse perspiration. Methods for determining diaphoresis in a patient are well known to those skilled in the art.

As used herein, the term “diastolic blood pressure” refers to the pressure exerted on the vessel walls when the heart muscle relaxes between beats and is filling with blood. Diastolic blood pressure is usually the second or bottom number in a blood pressure reading. Methods for measuring diastolic blood pressure are well known to those skilled in the art.

As used herein, the term “dyspnea” refers to difficult or labored breathing or shortness of breath. Methods for determining dyspnea in a patient are well known to those skilled in the art.

By an “effective amount” or a “therapeutically effective amount” of a drug (such as, but not limited to, an inotropic drug, a vasodilator, a beta-blocker or combinations thereof, etc.) is meant a nontoxic but sufficient amount of the drug or exposure to the drug to provide the desired effect. The amount of drug that is “effective” will vary from patient to patient, depending on the age and general condition of the individual, the particular drug or drugs, and the like. Thus, it is not always possible to specify an exact “effective amount.” However, an appropriate “effective amount” in any individual case may be determined by those skilled in the art using routine experimentation.

The term “inotropic drug” or “positive inotropic drug” refers to a drug that increases the force of myocardial contractility, with or without other physiological effects, such as, but not limited to, calcium-sensitization, vasodilation, phosphodiesterase-inhibiting activity, etc. Inotropic drugs are well known in the art and include, but are not limited to, levosimendan, dopamine, dobutamine, inamrinone, milrinone, dopexamine, digoxin, enoximone, pimobendan and metabolites thereof.

The term “levosimendan” refers to any racemic mixture or enantiomer of levosimendan or a racemic mixture or enantiomer of the metabolites of levosimendan. The term “levosimendan” specifically refers to the (−)-enantiomer of [4-(1,4,5,6-tetrahydro-4-methyl-6-oxo-3-pyridazinyl)phenyl]hydrazono]propanedinitrile. The term also is intended to encompass combinations of levosimendan and its metabolites. A metabolite of levosimendan is, for example, (R)—N-[4-(1,4,5,6-tetrahydro-4-methyl-6-oxo-3-pyridazinyl)phenyl]acetamide.

As used herein, the term “mean arterial pressure” or “MAP” refers to the average blood pressure in a patient. MAP is used to assess the hemodynamic status of a patient. More specifically, it is considered the perfusion pressure seen by organs in the body. Formulas for calculating MAP are well known to those skilled in the art. An example of a formula that can be used to calculate MAP is:

MAP=⅔ diastolic blood pressure+⅓ systolic blood pressure

As used herein, the phrase “New York Heart Association (NYHA) Classification” refers to the following functional and therapeutic classification for CHF patients:
Class I: patients with no limitation of activities; they suffer no symptoms for ordinary activities.
Class II: patients with slight, mild limitation of activity; they are comfortable with rest or with mild exertion.
Class III: patients with marked limitation of activity; they are comfortable only at rest. Patients in the early stage of Class III (less severe symptoms) are sometimes classified as being in Class IIIa. Patients in the late stage of Class III (more advanced symptoms) are sometimes classified as being in Class IIIb.
Class IV: patients who should be at complete rest, confined to bed or chair; any physical activity brings on discomfort and symptoms occur at rest.

The term “patient” refers to an animal, preferably a mammal, including a human or non-human. The terms subject and patient may be used interchangeably herein.

As used herein, the term “pulmonary edema” refers to the abnormal accumulation of extravascular fluid within the lung. Pulmonary edema typically results in the patient experiencing difficulty breathing. Methods for determining pulmonary edema in a patient are well known to those skilled in the art.

As used herein, the term “systolic blood pressure” refers to the pressure exerted on the walls of the arteries during the contraction phase of the ventricles of heart. Systolic blood pressure is usually the first or top number in a blood pressure reading. Methods for measuring systolic blood pressure are well known to those skilled in the art.

As used herein, the term “tachypnea” refers to abnormally fast breathing or a respiratory rate that is rapid. Methods for determining tachypnea in a patient are well known to those skilled in the art.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

The term “vasodilator(s)” refers to a drug that opens the arteries and veins thereby reducing the heart's workload and allowing, more blood to reach the tissues. Several types of vasodilators are known in the art and include, but are not limited to, hydralazine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate, isosorbide mononitrate and metabolites thereof.

The Present Invention

As mentioned briefly above, the present invention relates to the discovery that certain blood pressure measurements (i.e., measurements of systolic blood pressure, diastolic blood pressure or mean arterial pressure) obtained from patients diagnosed with congestive heart failure provide useful clinical information. Specifically, the inventors of the present invention have discovered that the systolic blood pressure, diastolic blood pressure or mean arterial pressure of a CHF patient, which is at, above or below at least one predetermined criterion can be used to predict an increased or decreased likelihood of an improved clinical outcome in said patients. Additionally, this information can be used to determine whether or not to begin a treatment regimen with certain drugs, whether to continue or discontinue a specific treatment regimen with certain drugs and whether or not to optimize said treatment regimen with certain drugs.

More specifically, the inventors have discovered that useful information can be obtained from the blood pressure measurements of a patient diagnosed with CHF and particularly, when such measurements fall within at least one of the following predetermined criteria, namely, a systolic blood pressure that is less than, equal to or greater than about 100 mmHg, a diastolic blood pressure that is less than, equal to or greater than about 60 mmHg or a mean arterial pressure that is less than, equal to or greater than about 75 mmHg. Furthermore, such information can be used to establish a treatment regimen that will result in improved clinical outcomes over current standard of care regimens.

In particular, the inventors have found that CHF patients having a systolic blood pressure equal to or greater than about 100 mmHg, a diastolic blood pressure equal to or greater than about 60 mmHg or a mean arterial pressure equal to or greater than about 75 mmHg tend to exhibit an improved clinical outcome as their blood pressure increases. These CHF patients are likely to experience a fewer number of morbidities (particularly when compared to CHF patients having a systolic blood pressure that is less than about 100 mmHg, a diastolic blood pressure that is less than about 60 mmHg or a mean arterial pressure that is less about 75 mmHg), such as, but not limited to, a shorter duration of hospitalization, improved or unchanged dyspnea and/or tachypnea, improved or unchanged diaphoresis, improved or unchanged cyanosis and/or improved or unchanged mental status. Additionally, these patients have a lower risk of mortality (particularly when compared to CHF patients having a systolic blood pressure that is less than about 100 mmHg, a diastolic blood pressure that is less than about 60 mmHg or a mean arterial pressure that is less about 75 mmHg).

Moreover, the inventors also found that CHF patients having a systolic blood pressure that is less than about 100 mmHg, a diastolic blood pressure that is less than about 60 mmHg and a mean arterial pressure that is less than about 75 mmHg tend to exhibit a decreased clinical outcome as their blood pressure decreases. These CHF patients are likely to experience a greater number of morbidities (particularly when compared to CHF patients having a systolic blood pressure that is equal to or greater than about 100 mmHg, a diastolic blood pressure that is equal to or greater than about 60 mmHg or a mean arterial pressure that is equal to or greater than about 75 mmHg), such as, but not limited to, a longer duration of hospitalization, worsening dyspnea and/or tachypnea, worsening diaphoresis, worsening cyanosis and/or worsening mental status. Additionally, these patients have a higher risk of mortality (particularly when compared to CHF patients having a systolic blood pressure that is equal to or greater than about 100 mmHg, a diastolic blood pressure that is equal to or greater than about 60 mmHg or a mean arterial pressure that is equal to or greater than about 75 mmHg).

Additionally, the inventors also discovered that CHF patients having a systolic blood pressure less than about 100 mmHg, a diastolic blood pressure less than about 60 mmHg and a mean arterial pressure than about 75 mmHg and that were being treated with at least one inotropic drug or at least one vasodilator exhibited a decreased clinical outcome when compared to CHF patients having a systolic blood pressure less than about 100 mmHg, a diastolic blood pressure less than about 60 mmHg and a mean arterial pressure less than about 75 mmHg and that were not being treated with at least one inotropic drug or at least one vasodilator. These CHF patients are likely to experience a greater number of morbidities (particularly when compared to CHF patients having a systolic blood pressure that is less than about 100 mmHg, a diastolic blood pressure that is less than about 60 mmHg or a mean arterial pressure that is less than about 75 mmHg and who are not being treated with at least one inotropic drug or at least one vasodilator), such as, but not limited to, a longer duration of hospitalization, worsening dyspnea and/or tachypnea, worsening diaphoresis, worsening cyanosis and/or worsening mental status. Additionally, these patients have a higher risk of mortality (particularly when compared to CHF patients having a systolic blood pressure that is less than about 100 mmHg, a diastolic blood pressure that is less than about 60 mmHg or a mean arterial pressure that is less than about 75 mmHg and who are not being treated with at least one inotropic drug or at least one vasodilator).

As can be appreciated by one skilled in the art, the above-described useful information can be derived from just a single blood pressure measurement taken from a CHF patient at any point time. However, multiple blood pressure measurements and averages of said measurements can also provide useful clinical information and are also contemplated as being within the scope of the present invention.

As can further be appreciated by those skilled in the art, the measurements resulting from the instruments used to obtain a systolic blood pressure reading or a diastolic blood pressure reading inherently contain a certain amount of error. Therefore, as used herein, the term “about” as it relates to the systolic and diastolic blood pressure reading, encompasses blood pressure readings of ±10%, preferably, ±5%. For example, the phrase “a systolic blood pressure less than, equal to or greater than about 100 mmHg” encompasses systolic blood pressure readings between 90 mmHg and 110 mmHg but preferably between 95 mmHg and 105 mmHg. One skilled in the art would recognize that the error guidelines (the ±10%, preferably, ±5%) referred to herein are simply to be used as a guideline. Those skilled in the art would recognize that additional variation in a blood pressure reading could be introduced based upon the CHF patient's condition and the actual blood pressure reading itself (It is well known in the art that the higher a blood pressure reading, the more accurate the reading. For example, a systolic blood pressure reading of 130 mmHg is considered to be more accurate than a systolic blood pressure reading of 90 mmHg).

In one embodiment, the present invention relates to using at least one of the predetermined criteria discussed herein in a method for predicting an improved clinical outcome in a patient diagnosed with CHF. More specifically, the inventors have found that patients diagnosed with CHF and who have a systolic blood pressure that is equal to or greater than about 100 mmHg, a diastolic blood pressure that is equal to or greater than about 60 mmHg or a mean arterial pressure that is equal to or greater than about 75 mmHg have an increased likelihood of experiencing an improved clinical outcome, particularly when compared to CHF patients who have a systolic blood pressure that is less than about 100 mmHg, a diastolic blood pressure that is less than about 60 mmHg or a mean arterial pressure that is less than about 75 mmHg. An increased likelihood of an improved clinical outcome refers to the fact that these CHF patients are likely to experience a fewer number of morbidities (particularly when compared to CHF patients having a systolic blood pressure that is less than about 100 mmHg, a diastolic blood pressure that is less than about 60 mmHg or a mean arterial pressure that is less about 75 mmHg), such as, but not limited to, a shorter duration of hospitalization, improved or unchanged dyspnea and/or tachypnea, improved or, unchanged diaphoresis, improved or unchanged cyanosis and/or improved or unchanged mental status. Additionally, these patients have a lower risk of mortality (particularly when compared to CUP patients having a systolic blood pressure that is less than about 100 mmHg, a diastolic blood pressure that is less than about 60 mmHg or a mean arterial pressure that is less about 75 mmHg).

In contrast, CHF patients that have a systolic blood pressure that is less than about 100 mmHg, a diastolic blood pressure that is less than about 60 mmHg or a mean arterial pressure that is less than about 75 mmHg have a decreased likelihood of experiencing an improved clinical outcome. Specifically, these patients are likely to experience a greater number of morbidities (particularly when compared to CHF patients having a systolic blood pressure that is equal to or greater than about 100 mmHg, a diastolic blood pressure that is equal to or greater than about 60 mmHg or a mean arterial pressure that is equal to or greater than about 75 mmHg), such as, but not limited to, a longer duration of hospitalization, worsening dyspnea and/or tachypnea, worsening diaphoresis, worsening cyanosis and/or worsening mental status. Additionally, these patients have a higher risk of mortality (particularly when compared to CHF patients having a systolic blood pressure that is equal to or greater than about 100 mmHg, a diastolic blood pressure that is equal to or greater than about 60 mmHg or a mean arterial pressure that is equal to or greater than about 75 mmHg).

The above information can be used in a method to predict whether a patient diagnosed with CHF will experience an improved clinical outcome. Such a method involves obtaining or measuring the systolic blood pressure, diastolic blood pressure or mean arterial pressure of a patient with CHF. Methods of measuring blood pressure are well known in the art. Once the systolic blood pressure, diastolic blood pressure or mean arterial pressure is obtained, an analysis is made, such as by a clinician, to determine where that blood pressure measurement falls or fits in relationship to at least one of the predetermined criteria described herein. Once this determination is made, a prediction can be made, such as by a clinician, as to whether or not there is an increase or decreased likelihood that the patient will experience an improved clinical outcome.

For example, the systolic blood pressure of a patient diagnosed with CHF and who is currently hospitalized, is obtained by a clinician. The systolic blood pressure of the patient is 93 mmHg. Based upon the predetermined criteria described above, the clinician would be able to predict that based on this systolic blood pressure of 93 mmHg that the patient has a decreased likelihood of an improved clinical outcome. In contrast, if the systolic blood pressure of this patient was determined to be 107 mmHg, the clinician would be able to predict that based on this systolic blood pressure that the patient has an increased likelihood of an improved clinical outcome.

In a second embodiment, the present invention relates to using at least one of the predetermined criteria discussed herein to determine whether or not to begin or commence a course of drug therapy in a patient diagnosed with CHF. More specifically, the present inventors have discovered that patients diagnosed with CHF and who have a systolic blood pressure that is less than about 100 mmHg, a diastolic blood pressure that is less than about 60 mmHg or a mean arterial pressure that is less than about 75 mmHg and that have been treated with at least one inotropic drug, at least one vasodilator or combinations thereof, experience a decreased likelihood of an improved clinical outcome. With respect to the decreased likelihood of an improved clinical outcome, these patients are likely to experience a greater number of morbidities such as, but not limited to, a longer duration of hospitalization, worsening dyspnea and/or tachypnea, worsening diaphoresis, worsening cyanosis and/or worsening mental status (particularly when compared to CHF patients having a systolic blood pressure that is equal to or greater than about 100 mmHg, a diastolic blood pressure that is equal to or greater than about 60 mmHg or a mean arterial pressure that is equal to or greater than about 75 mmHg). Additionally, these patients have a higher risk of mortality (particularly when compared to CHF patients having a systolic blood pressure that is equal to or greater than about 100 mmHg, a diastolic blood pressure that is equal to or greater than about 60 mmHg or a mean arterial pressure that is equal to or greater than about 75 mmHg).

In contrast, the inventors have also discovered that patients diagnosed with CHF and that have a systolic blood pressure that is equal to or greater than about 100 mmHg, a diastolic blood pressure that is equal to or greater than about 60 mmHg or a mean arterial pressure that is equal to or greater than about 75 mmHg and that have been treated with at least one inotropic drug, at least one vasodilator or combinations thereof do not experience a decreased likelihood of an improved clinical outcome. Rather, many such CHF patients experience an increased likelihood of an improved clinical outcome when treated with at least one inotropic drug, at least one vasodilator of combinations thereof. An increased likelihood of an improved clinical outcome refers to the fact that these CHF patients are likely to experience a fewer number of morbidities such as, but not limited to, a shorter duration of hospitalization, improved or unchanged dyspnea and/or tachypnea, improved or unchanged diaphoresis, improved or unchanged cyanosis and/or improved or unchanged mental status (particularly when compared to CHF patients having a systolic blood pressure that is less than about 100 mmHg, a diastolic blood pressure that is less than about 60 mmHg or a mean arterial pressure that is less than about 75 mmHg). Additionally, these patients have a lower risk of mortality (particularly when compared to CHF patients having a systolic blood pressure that is less than about 100 mmHg, a diastolic blood pressure that is less than about 60 mmHg or a mean arterial pressure that is less than about 75 mmHg).

The above information can be used in a method to determine whether or not to commence or begin a course of monotherapy with an inotropic drug or vasodilator in a patient diagnosed with CHF. Such a method involves obtaining or measuring the systolic blood pressure, diastolic blood pressure or mean arterial pressure of a patient with CHF. Methods of measuring blood pressure are well known in the art. Once the systolic blood pressure, diastolic blood pressure or mean arterial pressure is obtained, an analysis is made, such as by a clinician, to determine where the blood pressure measurement falls or fits in relationship to at least one of the predetermined criteria described above. Once this determination is made, a further determination is made, such by a clinician, as to whether or not a course of monotherapy should or should not be commenced or begun in the patient. If a determination is made that a course of monotherapy should be commenced, the patient can be administered an effective amount of an inotropic drug or a vasodilator. The inotropic drug or vasodilator selected for administration will at the discretion of the clinician. The inotropic drug or vasodilator to be used in the monotherapy can be administered using any routes known in the art, such as, but not limited to, orally, buccally, intravenously, intramuscularly, subcutaneously, by inhalation, etc.

For example, the systolic blood pressure of a patient diagnosed with CHF and who is currently hospitalized, is obtained by a clinician. The systolic blood pressure is 91 mmHg. Based upon the predetermined criteria described above, the clinician would decide not to begin a course of monotherapy with an inotropic drug or vasodilator in the patient based on this blood pressure measurement. In contrast, if the systolic blood pressure of this patient was 109 mmHg, the clinician would decide that a course of monotherapy with an inotropic drug or vasodilator could begin in said patient based on this blood pressure measurement. The clinician could intravenously administer an effective amount of an inotropic drug, such as levosimendan, or a vasodilator, such as primacor, to the CHF patient.

Alternatively, the above information can be used in a method to determine whether or not to begin a course of combination therapy with at least one inotropic drug, at least one vasodilator or combinations thereof in a patient diagnosed with CHF. As with method described above regarding monotherapy, such a method involves obtaining or measuring the systolic blood pressure, diastolic blood pressure or mean arterial pressure of a patient with CHF. Methods of measuring blood pressure are well known in the art. Once the systolic blood pressure, diastolic blood pressure or mean arterial pressure is obtained, an analysis is made, such as by a clinician, to determine where the blood pressure measurement falls or fits in relationship to at least one of the predetermined criteria described herein. Once this determination is made, a further determination is made, such by a clinician, as to whether or not a course of combination therapy should be commenced or begun in the patients. If a determination is made that a course of combination therapy should be commenced, the patient can be administered all effective amount of at least one inotropic drug, at least one vasodilator or combinations thereof. For example, the patient can be administered an effective amount of two different inotropic drugs (such as levosimendan and dobutamine), an effective amount of two different vasodilators (such as hydralazine and natrecor), or any effective amount of any number of combinations of one or more inotropic drugs and one or more vasodilators (such as levosimendan and hydralazine, levosimendan, dobutamine and natrecor, levosimendan, hydralazine and natrecor, etc.). The at least one inotropic drug, at least one vasodilator or combinations thereof selected for administration will at the discretion of the clinician. The at least one inotropic drug, at least one vasodilator or combinations thereof to be used in the combination can be administered using any routes or combination of routes known in the art, such as, but not limited to, orally, buccally, intravenously, subcutaneously, intramuscularly, by inhalation, etc.

For example, the diastolic blood pressure of a patient diagnosed with CHF and who is currently hospitalized, is obtained by a clinician. The diastolic blood pressure in the patient is 55 mmHg. Based upon the predetermined criteria described above, the clinician would decide not to begin a course of combination therapy with at least one inotropic drug, at least one vasodilator or combinations thereof in the patient based on this blood pressure measurement. In contrast, if the diastolic blood pressure of this patient was 67 mmHg, the clinician would decide that a course of combination therapy with at least one inotropic drug, at least one vasodilator or combinations thereof could begin in said patient based on this blood pressure measurement. The clinician may then administer intravenously an effective amount of levosimendan and an effective amount of hydralazine to the CHF patient.

Using the information discussed above in the second embodiment, in a third embodiment, the present invention relates to a method for determining whether to continue or discontinue a course of monotherapy with an inotropic drug or vasodilator or whether to continue or discontinue a course of combination therapy with at least one inotropic drug, at least one vasodilator or combinations thereof in a patient diagnosed with CHF and who is currently receiving said monotherapy or combination therapy. The amount of time that the CUP patient has been receiving said monotherapy or combination therapy is not critical to this method.

In this embodiment, the method involves obtaining or measuring the systolic blood pressure, diastolic blood pressure or mean arterial pressure of a patient with CHF and who is currently being treated with an inotropic drug or vasodilator (monotherapy) or at least one inotropic drug, at least one vasodilator or combinations thereof (combination therapy). Methods of measuring blood pressure are well known in the art. Once the systolic blood pressure, diastolic blood pressure or mean arterial pressure is obtained, an analysis is made, such as by a clinician, to determine where the blood pressure measurement falls or fits in relationship to at least one of the predetermined criteria described herein. Once this determination is made, a further determination is made, such by a clinician, as to whether to continue or discontinue the course of monotherapy (with the inotropic drug or vasodilator) or combination therapy (at least one inotropic drug, at least one vasodilator or combinations thereof) in said patient. If a determination is made that a course of monotherapy or combination therapy should be continued, the patient will continue to receive the currently existing monotherapy or combination therapy subject to any changes or modifications that the clinician may, in his or her discretion, wish to make. If a determination is made that the course of monotherapy or combination therapy should be discontinued (stopped or terminated), then, the patient immediately ceases receiving any further monotherapy or combination therapy. Certainly, at a later period in time and depending on the health of the patient, the clinician can use the method described in the second embodiment herein to determine whether or not to again begin a course of monotherapy or combination therapy in said patient.

For example, the mean arterial pressure of a patient diagnosed with CHF, who is currently hospitalized and is currently being treated with an effective amount of levosimendan and dobutamine, is obtained by a clinician. The mean arterial pressure of this patient is 65 mmHg. Based upon the predetermined criteria described above, the clinician would decide to discontinue the patient's treatment with levosimendan and dobutamine based on this blood pressure measurement. In contrast, if the mean arterial pressure of this patient was 84 mmHg, the clinician would decide that the current course of treatment with levosimendan and dobutamine should be continued in the patient based on this blood pressure measurement.

Using the information discussed above in the second embodiment, in a fourth embodiment, the present invention relates to a method for optimizing an existing or current drug treatment regimen in a patient diagnosed with CHF. The existing or current drug treatment regimen of the CHF patient comprises the administration to the patient of an effective amount of at least one inotropic drug, at least one vasodilator, at least one beta-blocker or combinations thereof. The amount of time that the CHF patient has been receiving said treatment regimen is not critical to this method.

In this fourth embodiment, the method involves obtaining or measuring the systolic blood pressure, diastolic blood pressure or mean arterial pressure of a patient with CHF and who is currently receiving a treatment regimen that includes at least one inotropic drug, at least one vasodilator, at least one beta-blocker or combinations thereof. Methods of measuring blood pressure are well known in the art. Once the systolic blood pressure, diastolic blood pressure or mean arterial pressure is obtained, an analysis is made, such as by a clinician, to determine where the blood pressure measurement falls or fits in relationship to at least one of the predetermined criteria described herein. Once this determination is made, a further determination is made, such by a clinician, to optimize the treatment regimen to increase the likelihood of an improved clinical outcome in the patient. An increase likelihood of an improved clinical outcome refers to the fact that these CHF patients would be likely to experience a fewer number of morbidities such as, but not limited to, a shorter duration of hospitalization, improved or unchanged dyspnea and/or tachypnea, improved or unchanged diaphoresis, improved or unchanged cyanosis and/or improved or unchanged mental status (particularly when compared to CHF patients having a systolic blood pressure that is less than about 100 mmHg, a diastolic blood pressure that is less than about 60 mmHg or a mean arterial pressure that is less about 75 mmHg). Additionally, these patients would have a lower risk of mortality (particularly when compared to CHF patients having a systolic blood pressure that is less than about 100 mmHg, a diastolic blood pressure that is less than about 60 mmHg or a mean arterial pressure that is less about 75 mmHg).

Alternatively, the method herein may involve obtaining or measuring the systolic blood pressure, diastolic blood pressure or mean arterial pressure of a patient with CHF and who is currently receiving a treatment regimen that includes at least one inotropic drug, at least one vasodilator, at least one beta-blocker or combinations thereof at more than one period in time. Once at least two (2) systolic blood pressure, diastolic blood pressure or mean arterial pressure measurements have been obtained (preferably after some period of time has passed from the time the first systolic blood pressure, diastolic blood pressure or mean arterial pressure an analysis is made (such as, but not limited to 15 minutes, 30 minutes, 45 minutes, 60 minutes, 90 minutes, etc.)), such as by a clinician, the clinician will determine the size of the change between the last blood pressure measurement obtained from said CHF patient and the first blood pressure measurement obtained from said patient (also known as the “delta”). If the delta is at least 10%, a further determination is made, such by a clinician, to optimize the treatment regimen to increase the likelihood of an improved clinical outcome in the patient.

For example, the first systolic blood pressure reading of a CHF patient at 9:00 am is 110 mmHg. At 10:30 am the second systolic blood pressure reading of the same CHF patient is 95 mmHg. The delta change is 15 mmHg in 1½ hours and this change is at least 10% of the first systolic blood pressure reading taken at 9:00 am in this patient. Based on this information, the clinician would optimize this CHF patient's treatment regiment in order to increase the likelihood of an improved clinical outcome for this patient.

For example, the first systolic blood pressure reading of a CHF patient at 9:00 am is 120 mmHg. At 10:30 am, the second systolic blood pressure reading of the same CHF patient is 112 mmHg. At noon, the third systolic blood pressure reading of the same CHF patient is 90 mmHg. The delta change is 30 mmHg in 3 hours and this change is at least 10% of the first systolic blood pressure reading taken at 9:00 am in this patient. Based on this information, the clinician would optimize this CHF patient's treatment regiment in order to increase the likelihood of an improved clinical outcome for this patient.

Any optimization of the treatment regimen that increases the likelihood of an improved clinical outcome for the CHF patient described herein is contemplated by the method of the present invention. For example, the optimization of the treatment regimen might involve a decision by the clinician to discontinue treating the patient with the at least one inotropic drug, at least one vasodilator, at least one beta-blocker or combinations thereof. Alternatively, the optimization of the treatment regimen might involve a decision by the clinician to substitute an effective amount of a different inotropic drug, a different vasodilator, a different beta-blocker or combinations thereof that are not currently being used in the existing treatment regimen for an effective amount of at least one inotropic drug, at least one vasodilator, at least one beta-blocker or combinations thereof in the existing treatment regimen. Alternatively, the optimization of the treatment regimen might involve a decision by the clinician to add an effective amount of at least one different inotropic drug, at least one vasodilator, at least one beta-blocker or combinations thereof that are not currently being used in the existing treatment regimen.

For example, the systolic blood pressure of a patient diagnosed with CHF, who is currently hospitalized and is currently being treated with an effective amount of a regimen of dobutamine and propranolol, is obtained by a clinician. The systolic blood pressure of this patient is 88 mmHg. Based upon the predetermined criteria described above, the clinician would determine to discontinue the patient's treatment with dobutamine and propranolol based on this blood pressure measurement. In contrast, if the systolic blood pressure of this patient was 112 mmHg, the clinician could determine to optimize the patient's treatment regimen by adding levosimendan to the treatment regimen based on this blood pressure measurement. Alternatively, the clinician could determine to optimize the patient's treatment regimen by discontinuing the patient's treatment with dobutamine in the patient's treatment regimen and replacing it with levosimendan. Alternatively, the clinician could determine to optimize the patient's treatment regimen by discontinuing the patient's treatment with dobutamine and propranolol and replacing it with a treatment regimen comprising levosimendan, hydralazine and esmolol.

In another embodiment, the invention includes any of the methods described herein and communication media or labeling describing a method or the results of a study as disclosed herein. The media can include one or more of the group consisting of a package insert, a quick-reference card, and printed packaging material, which are preferably physically bound together. The results of a clinical study, such as without limitation, the clinical studies described herein, evaluates the effect of the methods on a population of patients. Alternatively, the packaging material instead can identify a source of electronic mediate that discloses the results or a summary of the clinical study.

Methods of generating labeling which describes or discloses a method or a clinical study result are well known to those of ordinary skill in the art.

The invention also provides a business method, such as a method of promoting the sale or use of a drug, for example, levosimendan, which is used in the treatment of congestive heart failure. The method includes at least providing such a drug, preferably levosimendan, in a pharmaceutically-acceptable form for administration to a patient, and encouraging a decision maker to refer to printed, graphic, or electronic media disclosing a method as described herein or a result of a clinical study of the effects of administering such drug to a patient. The decision maker can be any suitable person such as a patient, a health care provider; a health care payer, an insurer, an employee or official of a formulary board, or an employee or official of a medical welfare program. The decision maker can be an actual person or a “legal person” such as an agency or corporation, so long as the person exercises some control or influence over the medical process or the insurance process relating to the purchase of pharmaceutical products.

The business method also can be practiced by providing a customer or medical decision maker with printed, graphic, or electronic media disclosing a method as provided herein or a clinical study of the effect of administering a drug for the treatment of congestive heart failure. Preferably, the drug is levosimendan.

Alternatively, one also can practice the method also by distributing labeling or causing labeling to be distributed. Such labeling includes but is not limited to printed, graphic, or electronic media that describes a method as disclosed herein or the results of a clinical study of the effects of administering a drug for the treatment of congestive heart failure. For example, small (e.g., weighing less than 1 pound) items can be distributed bearing a web-site universal resource locator (URL), wherein the website contains a copy of the a study report, a summary of a study and/or its results, data summarized from the study report, or an editorial referring to the study and/or its results. The small items can include, but are not limited to, notepads, pens, calendars, pins, clothing items, pill counters, toys and novelties, bags (preferably plastic bags), and business cards, brochures, booklets, mailing pieces, detailing pieces, file cars, bulletins, price lists, catalogs, house organs or newsletters, letters, motion picture films, film strips, lantern slides, sound recordings, exhibits, literature, and reprints and similar pieces of printed, audio, or visual matter descriptive of the clinical study, published (for example, the :Physicians Desk Reference”) for use by medical practitioners, pharmacists, or nurses.

Further, the media can be distributed in connection with a medical education seminar, such as for example, a so-called continuing medical education (CME) seminar. Media distributed in connection with CME can include copies of presentation materials, advertisements for the CME event, and other materials.

The media also can be distributed to a sales representative whose duties include the sale of a drug for the treatment of congestive heart failure. The sales representative can, but need not further distribute the information in tangible form.

The method also provides a method of selling or promoting a drug for the treatment of congestive heart failure that comprises providing to a person information (such as a method as described herein), data, or a summary of results of a study and compensating the person according to the level of sales, consumption, distribution, or prescriptions written for such a drug in a group of consumers, physicians, pharmacists, or patients, or in a geographical region. In a preferred embodiment, the information is in tangible form, and more preferably, contains a presentation of facts or strategies for motivating the increased distribution of the drug.

By way of example, and not of limitation, examples of the present invention will now be given.

### EXAMPLE 1

### The REVIVE I Study and REVIVE II Study

Background of the Study

The purpose of this study was to evaluate the efficacy of a 24-hour infusion of levosimendan compared with placebo in the treatment of patients hospitalized with decompensated chronic heart failure. This multicenter (there were 97 enrolling centers in the United States, Australia and Israel) confirmatory Phase 3 study was designed to demonstrate the superiority of levosimendan compared to placebo, based on the evaluation and disposition of patients according to a clinical composite primary endpoint as the primary objective.

Objectives of the Study

The primary objective of this study was to identify the number of patients improved a each of the following time points: 6 hours, 24 hours, and 5 days or worsened at any time up to 5 days after the start of the study drug infusion using a composite scale.

The secondary objectives of this study included the following:
Time to death or ‘worsening’ heart failure during the first 31 days after the start of the study drug infusion.
Patient's Global Assessment at 6 hours after the start of the study drug infusion.
Patient's Dyspnea Assessment at 6 hours after the start of the study drug infusions
New York Heart Association (NYHA) Classification at 5 days after the start of the study drug infusion.
Change in plasma Brain Natriuretic Peptide (BNP) concentration from baseline to 24 hours after the start of the study drug infusion.
The duration of initial hospitalization after the start of the study drug infusion, defined as days alive and out of hospital during the first 14 days,
‘All-cause mortality’ during the 90 days following the start of the study drug infusion.

The total number of subject enrolled in this study was 700 (approximately 350 in each treatment group, 100 patients in REVIVE I (defined below in the “Methodology Used in the Study”) and 600 patients in REVIVE II (defined below in the “Methodology Used in the Study”).

Methodology Used in the Study

This study was a double-blind, placebo-controlled study with parallel group dosing in patients hospitalized for acute decompensation of heart failure. The treatment period was maximally 24 hours for each subject with at least 90 days follow-up after the start of the study drug infusion. The primary endpoint assessed efficacy at each of the following time points: 6 hours, 24 hours, and 5 days using a composite scale incorporating patients' global assessment and criteria for worsening heart failure. Heart failure symptoms were assessed independently by both the patient and physician at a number of time points up to 5 days after the start of the study drug infusion. Safety parameters such as adverse events, heart rate, blood pressure, and ECG were monitored frequently up to 31 days with continued recording of hospitalization and mortality extended to 90 days after the start of the study drug infusion. Survival was monitored for all patients from day 90 until the last patient underwent the final scheduled assessment. Before randomization, patients were able to receive any or all appropriate treatments for heart failure, including intravenous diuretics, vasodilators, dobutamine or dopamine (but not amrinone or milrinone). Intravenous treatment ongoing at the start of the study drug infusion were continued along with any other treatments received by the patient before randomization although, IV continuous diuretics, inotropes, and vasodilators had to have been at a stable dose for at least 2 hours prior to baseline. Randomization was centralized and stratified for concurrent IV inotrope and/or vasodilator use to ensure even distribution of these patients between treatment groups. Following randomization and initiation of the study drug infusion, the investigator was entitled to treat the patient according to his/her clinical judgment but each time one of the defined ‘rescue’ therapies was started or the dose increased, the investigator had to state whether it was for worsening heart failure (and document such worsening) or specify another reason. Patients with documented worsening heart failure treated with a rescue therapy were considered to meet the criteria for worsening in the primary endpoint. Use of a rescue therapy not for worsening heart failure (e.g., IV diuretic for ongoing diuresis) did not constitute worsening. Right heart catheterization was not mandated or prohibited, but was carried out according to the judgment of the patient's physician. Patient visits took place on days 5, 31, and 90 with telephone contacts on days 14, 45, 60, and 75 after the start of the study drug infusion. If the patient was discharged before day 7, adverse event inquiries were conducted daily by telephone up to day 7. Follow-up after day 90 was generally by telephone. An interim analysis was performed after 100 patients had completed the study (to 90 days) (REVIVE I) to check the assumptions and definitions used for the primary endpoint and the sample size calculation. The data from the first 100 patients was analyzed and reported separately from the main trial. For clarity the first 100 patient trials was termed “REVIVE I” with the remainder of the trial termed “REVIVE II”.

Diagnosis and Inclusion/Exclusion Criteria

All patients had been hospitalized with a primary or secondary admitting diagnosis of worsening heart failure and continued to have symptoms at rest despite treatment with IV diuretic therapy.

Inclusion Criteria:
1. Written, signed and dated informed consent by the patient or the patient's legally authorized representative
2. Male and female patients over 18 years of age. Females of childbearing potential had to have a negative pregnancy test and had to refrain from breastfeeding. Patients who were postmenopausal (two years since last menstrual cycle), surgically sterilized, or who had undergone a hysterectomy were considered not to be of childbearing potential.
3. Hospitalized with a primary or secondary diagnosis at admission of worsening heart failure within the 48 hours prior to the start of the study drug infusion. Symptoms of worsening heart failure must have been treated with IV diuretics. Patients who had been hospitalized more than 48 hours could be enrolled if they failed to improve clinically to treatments administered during the first 48 hours or if (following initial improvement) their clinical status deteriorated either spontaneously or following the withdrawal of intravenous medications. Infusion rates for continuous IV diuretics, inotropes, and vasodilators had to have been unchanged for at least 2 hours prior to baseline.
4. Left ventricular ejection fraction less than or equal to 35% as assessed using echocardiography, radionuclide ventriculography, or contrast angiography within the previous 12 months.
5. Dyspnea at rest at both screening and baseline, as assessed by the patient.

Exclusion Criteria:
1. Severe obstruction of ventricular outflow tracts such as hemodynamically significant uncorrected primary valve disease and restrictive or hypertrophic cardiomyopathy.
2. Patients scheduled to receive angioplasty, cardiac surgery, a LV assist device or a heart transplant within 3 months after randomization.
3. Patients who had undergone cardioversion during the 4 hours prior to baseline or were expected to undergo cardioversion in the 5 days after baseline.
4. Patients who had undergone a cardiac resynchronization procedure within the 30 days of screening or were expected to undergo such a procedure within 3 months.
5. Patients who had received an IV diuretics dose (or change in dose of a continuous diuretic infusion) within 2 hours of the baseline assessments.
6. Patients who were intubated or otherwise not able to comply with the pre-study assessments.
7. Stroke or TIA within 3 months prior to randomization
8. Systolic blood pressure 90 mmHg or less at screening or baseline.
9. Heart rate 120 bpm or greater, persistent for at least 5 minutes at screening or baseline.
10. Serum potassium less than 3.5 mmol/l or greater than 5.4 mmol/l.
11. Angina pectoris during the 6 hours before baseline.
12. Administration of amrinone or milrinone within 24 hours before start of study drug infusion.
13. Hypersensitivity to levosimendan or any of the excipients.
14. A history of Torsades de Pointes.
15. Severe renal insufficiency (serum creatinine>450 μmol/L [5.0 mg/dL]) or on dialysis.
16. Significant hepatic impairment or elevation of liver enzymes to 5 times the upper limit of normal.
17. Acute bleeding or severe anemia (hemoglobin<10 g/dL or blood transfusion during current admission) or acute decompensation due to an active infection. Patients with low hemoglobin between 9-10 g/dL could be enrolled provided there was no evidence of bleeding, no intention to transfuse blood, no identified cause for anemia other than renal insufficiency and if the severity of anemia was longstanding (documented hemoglobin+/−1 g/dL of screening value>30 days prior).
18. History of severe chronic obstructive pulmonary disease or unstable bronchial asthma as evidenced by e.g. CO₂ retention or ongoing use of oral, intravenous or intramuscular steroids
19. Participation in a clinical trial with any experimental treatment within the last 30 days or patients previously entered into the trial.

Dose

The levosimendan and placebo were administered intravenously. The placebo was diluted and infused intravenously according to the same schedule as the levosimendan (see below). The duration of the treatment was 24 hours. The specific details as to the schedule of intravenous administration of levosimendan is provided below:

1. A total loading dose of 12 μg/kg administered over 10 minutes (6 μg/kg for patients on concomitant IV vasodilators or inotropes at the start of the study drug infusion).

2. A continuous infusion of 0.1 μg/kg/min for the following 50 minutes.

3. If the dose was well tolerated, the infusion rate was increased to 0.2 μg/kg/min for a further 23 hours.

4. If the higher dose was not well tolerated it could be reduced to 0.1 μg/kg/min and then further to 0.05 μg/kg/min if required.

On completion of 24 hours the infusion was turned off abruptly.

Variables and Methods of Assessments

The study drug infusion was started on day 0. Active assessments took place daily during the initial hospitalization and at days 5, 31, and 90 after the start of the study drug infusion. Clinical status and NYHA heart failure classification were determined by telephone on days 14, 45, 60, and 75, unless the patient was still hospitalized. NYHA heart failure classification was also performed on days 5, 31, and 90 at the patient visit
1. A Global Assessment (with the patient lying flat) was completed by the patient and the physician as change from baseline at 6 (patient assessment only), 24, 48 hours and day 3, day 5, and on discharge if before day 5, after the start of the study drug infusion. In addition, patients completed a Global Assessment modified visual analogue scale at baseline and the same time points.
2. Dyspnea (with patient lying flat) was evaluated by the patient at screening, baseline, and the change at 6, 24, 48 hours and day 3, day 5, and on discharge if before day 5, after the start of the study drug infusion compared to baseline.
3. Hospitalization. Any calendar day, when the patient was either hospitalized or fulfilled either of the following criteria from the time of randomization to day 90 after the start of the study drug infusion or death, if sooner, was considered a hospitalization day:
a. The patient attended the emergency room for an emergent medical condition.
b. The patient received any IV medication for heart failure (including ambulatory dobutamine treatment).
4. Mortality. Date, time, location, and cause of death were recorded to day 90 after the start of the study drug infusion. In addition, patients were followed until the end of the study, i.e., last patient last visit. The investigator contacted the patient approximately every three months by telephone and indicated on the CRF whether the patient was alive or record the date, time, location, and cause of death.
5. Adverse events. An adverse event inquiry was performed before the administration of the study drug infusion, immediately after stopping the study drug infusion, daily up to day 7, and then on days 14 and 31 after the start of the study drug infusion. Only serious adverse events with at least a possible causality (or not assessable) were recorded between days 31 and 90.
6. Heart rate and blood pressure were recorded at screening, baseline, 10 minutes, 1, 2, 3, 4, 5, 6, 12, 18, 24, 30, 36, 42, and 48 hours and thereafter three times daily up to day 4 and then twice daily on day 5. If still hospitalized, they were recorded twice daily on day 6 and once on day 7. Blood pressure and heart rate were also measured at the day 31 visit. Any episode of hypotension (whether symptomatic or not) or heart rate>140 bpm was closely monitored until resolutions
7. A 12-lead electrocardiogram was recorded at screening, baseline, 24 hours, and days 3, 5, and 31 after the start of the study drug infusion. All recording was taken at the same time of day as the baseline trace where practical. A central laboratory performed the measurement of ECG intervals. QT was corrected for heart rate using standard formula.
8. Biochemistry and hematology laboratory tests were analyzed at a central laboratory performed at baseline, 24 hours, and days 3, 5, and 31 after the start of the study drug infusion. Eligibility was established from samples analyzed at the local laboratory within 12 hours of baseline. A pregnancy test was performed at screening for women of childbearing potential. Plasma BNP concentrations were measured at baseline, 24 hours, day 5 and day 31.
9. Concomitant medications, including oral diuretics, were recorded in detail up to and including day 7 and until hospital discharge if later than day 7. The investigator had to indicate whether starting or increasing the dose of any defined rescue therapy was for worsening heart failure or specify the alternative reason for use.

Patient Global Assessment

The assessment of the change from baseline in global assessment was made by the patient (Patient Global Assessment) on a seven-point scale (markedly improved, moderately improved, mildly improved, no change, mildly worse, moderately worse, markedly worse) at 6, 24 and 48 hours and Day 3 and Day 5. In order to standardize the assessment procedure the patient was instructed to lie flat in bed (a single pillow is permitted) to evaluate the current symptom severity at each and every assessment timepoint, if their condition allowed. If the patient was unable to lie flat for screening or baseline dyspnea assessments, then the degree of elevation actually used was recorded in the patient's notes and all subsequent global assessments performed in that exact same position. The Patient Global Assessment was based on the following questions:
With respect to your heart failure, how do you feel now compared with how you felt at the time of the start of the study drug infusion?

Evaluation

The clinical composite consists of three possible responses; improved, unchanged or worse.

Improved: a ‘moderate’ or ‘marked’ improvement in global assessment (as determined by the patient using the seven-point scale) at each of the following time points: 6 hours, 24 hours, and 5 days. However, patients were not considered improved if they fulfilled any of the criteria for being ‘worse’ at any time during the 5 days after the start of the study drug infusion.

### Unchanged: did not meet criteria for either improved or worse.

Worse: unresponsive or worsening heart failure within 5 days after the start of the study drug infusion, as manifested by:
Death from any cause
Worsening heart failure including one of more of the clinical features of:
   Pulmonary congestion 1—worsening dyspnea and/or tachypnea
      2—increased pulmonary edema
   or low-output syndrome
      3—diaphoresis
      4—cool extremities and cyanosis
      5—worsening renal function
      6—decreased mental status
and resulting in the use of rescue therapy for heart failure.
Persistent and unresponsive symptoms of heart failure at rest (reflecting pulmonary congestion or low output state or both) after the 24-hour clinical assessment and resulting in the use of rescue therapy for heart failure.
At least moderately worse on the patient global assessment (7-point scale) at either 6 hours, 24 hours or 5 days, even if no rescue therapy is given.

To be considered as worsening in the analysis of the primary endpoint, one or more of the criteria for worsening had to have been met and had to have been accompanied by new treatment with an intravenous medication or an increased dose of an infusion present at baseline (e.g., intravenous diuretic, intravenous vasodilator, or intravenous positive inotropic agent) or the introduction of a mechanical approach to heart failure (e.g., intra-aortic balloon pump, mechanical ventilator, ventricular assist device) given for worsening heart failure at any time up to the day 5 assessment, except where the patient recorded at least moderate worsening on the Patient Global Assessment as above, or in the case of death. Only patients who died, reported at least moderate worsening, or received a rescue therapy as stated above were considered worse for the purposes of the endpoint.

Statistical Methods

The null hypothesis was that the distribution of the clinical composite endpoint was equal between treatment groups.

The critical time of patient eligibility was just prior to the start of the study drug infusion and was deemed to have occurred at the time of treatment allocation.

The Intent-to treat (ITT)-population comprised all randomized patients. The efficacy of levosimendan compared to placebo was evaluated in this population. The clinical composite was compared between the treatment groups using the Cochran-Mantel-Haenszel row mean score test stratified by baseline IV medication group. A per-protocol analysis was performed if this population was smaller than 95% of the ITT population.

Time to death or worsening heart failure and all-cause mortality were tabulated by treatment group and analyzed using the Cox-proportional-hazards model with effects for treatment and baseline IV medication group. Kaplan-Meier curves were produced. The patients' global assessment at 6 hours, patient's dyspnea at 6 hours, and the NYHA classification at 5 days were tabulated by treatment group and analyzed using the Cochran-Mantel-Haenszel row mean score test stratified by baseline IV medication group. The duration of initial hospitalization was analyzed using the log rank test. The change in plasma BNP concentration was summarized by treatment group using descriptive statistics and analyzed using ANOVA or Kruskal-Wallis test, whichever was appropriate, adjusted by the baseline IV medication group.

Safety variables (ECG, blood pressure, heart rate, routine laboratory tests) were summarized by treatment group using descriptive statistics. Adverse events were coded in MedDRA and listed for each treatment group according to System Organ Class and Preferred Term.

Additional variables were evaluated using the ITT-set only and no sensitivity analyses performed. Additional variables were performed for exploratory purposes include:
Cardiovascular mortality during 90 days after the start of the study drug infusion.
Cardiovascular morbidity during the 31 day follow-up period.
Time to first re-hospitalization for worsening heart failure after initial discharge.
Change from baseline in patient and physician Global Assessment (7-point scale which used the same scale as the Patient Global Assessment but the scale was determined by a physician rather than by the patient) and patient's assessment of dyspnea.
Change from baseline in NYHA classification at days 14, 31, 45, 60, 75, and 90.
Change from baseline in BNP concentrations at day 5 and day 31.
Duration of initial hospitalization.
All-cause mortality during the 31 days after the start of the study drug infusion
‘Number of days alive out of hospital’ (DAOH) during the 31 and 90 days following the start of the study drug infusion.
Patient survival at study completion.
Total diuretic usage to day 5.

Additional Ad Hoc Variables

Additional ad hoc analyses included:
All-cause morbidity.
Location of care for the initial hospitalization up to day 31.
Effects on renal function and in patients with renal impairment.
Concomitant IV vasoactive medication or non-pharmacological interventions up to day 7.
Cardiovascular morbidity during the 90 day follow-up period.

The data from the first 100 patients were analyzed and reported separately from the main trial. On the basis of the REVIVE I data, the sample size for the REVIVE II study was confirmed and the final definition for the primary endpoint made. Some of the secondary efficacy endpoints were changed as appropriate.

Safety variables (ECG, blood pressure, heart rate, routine laboratory tests) were summarized by treatment group using descriptive statistics. Adverse events were coded in MedDRA and listed for each treatment group according to System Organ Class and Preferred Term.

Results of the REVIVE I and REVIVE II Studies

The size and scope of the REVIVE I study were intended to demonstrate the initial performance characteristics and functionality of the clinical composite endpoint. The analysis of this study was conducted to interpret the overall primary response by treatment arm in the context of the directionality of conventional metrics for heart failure patients as well as the pre-specified response (primary endpoint). This study was not designed to discern a statistically significant response of the primary endpoint.

The results in the 100-patient cohort examined in the REVIVE I study showed that levosimendan-treated subjects demonstrated a beneficial response over the placebo arm on a background of standard-of-care therapy for each patient. The concordance of the clinical composite response with the original anticipated response, in addition to the confirmation of the robustness of this data with multiple sensitivity analyses, indicated that this index was suitable for implementation in a larger, pivotal study, REVIVE II. Furthermore, the positive response to levosimendan, as assessed by the clinical composite endpoint, was congruent with the favorable responses measured by the patient-defined, physician-defined, laboratory, biomarker, and clinical response secondary endpoints of this study.

With respect to adverse events, the proportion of patients reporting adverse events during 31 days was similar in the two treatment groups: 96% in the levosimendan arm and 94% in the placebo arm. The most commonly reported events during 31 days in both groups included hypotension, headache, nausea, and dizziness, all of which occurred in a higher proportion of levosimendan-treated subjects. Mortality was low during the study both at 31 days (5%) and at 90 days (9%). The median time to death was 60 days in the levosimendan group and 27 days in the placebo group. The proportion of patients experiencing serious adverse events during the study was similar in both treatment groups, with no evidence of a safety profile different from that previously described for levosimendan.

The findings in REVIVE I provided the basis to continue with the proposed levosimendan clinical program defined as REVIVE II.

The objective of this study was to evaluate the efficacy of levosimendan as measured by a clinically relevant patient response endpoint (clinical composite). This was a randomized, multinational (USA, Australia, and Israel), double blind study of a 24-hour infusion of either-levosimendan or placebo added to standard-of-care in patients with acutely decompensated heart failure. The study had greater than 90% power to detect a statistically significant difference between treatment groups in the clinical composite endpoint at an alpha level of <0.05. Secondary endpoints included differences between treatment arms in the following metrics after the start of study drug infusion: patients' global assessment at 6 hours, patients' assessment of dyspnea at 6 hours, plasma brain natriuretic peptide (BNP) levels at 24 hours, the duration of initial hospitalization, and mortality during the 90 days following study drug infusion.

Similar to the REVIVE I study, the clinical composite endpoint was designed to capture patient-reported responses, as measured by the Patient Global Assessment score, in response to therapy. These were concurrently combined with clinically relevant events to categorize patients' outcomes in terms of “improved,” “unchanged,” or “worsened” status to ensure that the measured responses were clinically meaningful.

Demographics

Baseline demographics were similar in both groups indicating that the study population was well randomized as illustrated in Table 1, below.

TABLE 1
Demographics and Habits

| | | Levosimendan | | Placebo | |
|---|---|---|---|---|---|
| Variable | Statistics | N = 299 | | N = 301 | |
| Sex | Male | 219 | 73.2% | 216 | 71.8% |
| | Female | 80 | 26.8% | 85 | 28.2% |
| Age (Years) | Mean (SD) | 64 (15) | | 63 (15) | |
| Weight (kg) | Mean (SD) | 84 (23) | | 86 (22) | |
| Ethnic Origin | Asian | 6 | 2.0% | 2 | 0.7% |
| | Black | 85 | 28.4% | 61 | 20.3% |
| | Caucasian | 182 | 60.9% | 204 | 67.8% |
| | Hispanic | 24 | 8.0% | 27 | 9.0% |
| | Other | 2 | 0.7% | 7 | 2.3% |

| | | | |
|---|---|---|---|
| % Ejection | Mean (SD) | 23 (6.9) | 24 (7.4) |
| Fraction | | | |
| (Screening) | | | |
| Duration of Heart | Mean (SD) | 62 (62) | 60 (61) |
| Failure (Months) | | | |

The data in Tables 2 and 3 demonstrate similar exposure between the treatment arms.

TABLE 2
Extent of Exposure to Study Drug

| | | Levosimendan | | Placebo | |
|---|---|---|---|---|---|
| Variable | Statistics | N = 293 | | N = 294 | |
| Total duration | Mean (SD) | 23 (4.5) | | 23 (3) | |
| (hour) | | | | | |
| Duration of | Mean (SD) | 10 (3) | | 10 (1.4) | |
| loading dose (min) | | | | | |
| Loading dose | 12 mcg/kg/10 min | 214 | 73.0% | 211 | 71.8% |
| | 6 mcg/kg/min | 78 | 26.6% | 82 | 27.9% |
| | Other | 1 | 0.3% | 1 | 0.3% |

| | | | |
|---|---|---|---|
| Duration of | Mean (SD) | 23 (4.5) | 23 (3) |
| continuous infusion | | | |
| Continuous | Mean (SD) | 0.18 (0.03 | 0.19 (0.02) |
| dose (mcg/kg/min) | | | |

TABLE 3
Final Study Drug Dose Level at 24 Hours

| | |
|---|---|
| Levosimendan | Placebo |
| N = 293 | N = 294 |

| Dose Level | N | % | N | % |
|---|---|---|---|---|
| 0.05 mcg/kg/min | 9 | 3.1 | 4 | 1.4 |
| 0.1 mcg/kg/min | 18 | 6.1 | 12 | 4.1 |
| 0.2 mcg/kg/min | 211 | 72.0 | 243 | 82.7 |
| Discontinued | 55 | 18.8 | 35 | 11.9 |

Primary Endpoint

The results for the intent-to-treat (ITT) protocol-defined clinical composite endpoint are shown in Table 4, below. The net clinical benefit to acutely decompensated heart failure patients was demonstrated by the greater proportion of patients categorized as improved and the smaller proportion of patients worsened (p-value=0.015) in the levosimendan treatment group. This response, based on a stringent improvement in patients' status at 6 hour and then maintained through day 5 without worsening as assessed by the primary endpoint, indicates that levosimendan provides a rapid, recognizable and relevant benefit to heart failure patients.

TABLE 4
Protocol-Defined Clinical Composite
Intent-to-Treat

| | | |
|---|---|---|
| Levosimendan | Placebo | Overall |
| N = 299 | N = 301 | p-value |

| | | | | | |
|---|---|---|---|---|---|
| Improved | 58 | 19.4% | 44 | 14.6% | 0.015 |
| Unchanged | 183 | 61.2% | 175 | 58.1% | |
| Worse | 58 | 19.4% | 82 | 27.2% | |

p-value from Cochran-Mantel-Haenszel test from stratified model

The robustness of the primary endpoint response was confirmed by multiple sensitivity analyses, which consisted of: exclusion of worsening heart failure events due to symptoms alone, removing the 6-hour assessment from the endpoint requiring lower threshold for improvement at 6-hours, exclusion of IV diuretics as rescue therapy in the first 72-hours and using the per-protocol population in place of the intent-to-treat.

Secondary Endpoints

For the purposes of supporting the primary endpoint and overall objective of the study, the secondary endpoints were initially to be analyzed at a significance level of 0.050 without adjustment for multiplicity.

The hierarchical order of the secondary endpoints was pre-specified as follows:
1. Mean change in plasma B-Type Natriuretic Peptide (BNP) concentration from baseline to 24 hours after the start of the study drug infusion.
2. Patient Global Assessment at 6 hours after the start of the study drug infusion.
3. Patient Dyspnea Assessment at 6 hours after the start of the study drug infusions
4. The duration of initial hospitalization after the start of study drug infusion (defined as days alive and out of hospital during the first 14 days).
5. Time to death or ‘worsening’ heart failure during the first 31 days after the start of the study drug infusion.
6. New York Heart Association (NYHA) classification at Day 5 after the start of the study drug infusion.
7. ‘All-cause mortality’ during the 90 days following the start of the study drug infusion.

The results of the secondary endpoints for the REVIVE II are presented below in accordance with the pre-defined Statistical Analysis Plan as described above.

B-Type Natriuretic Peptide (BNP)

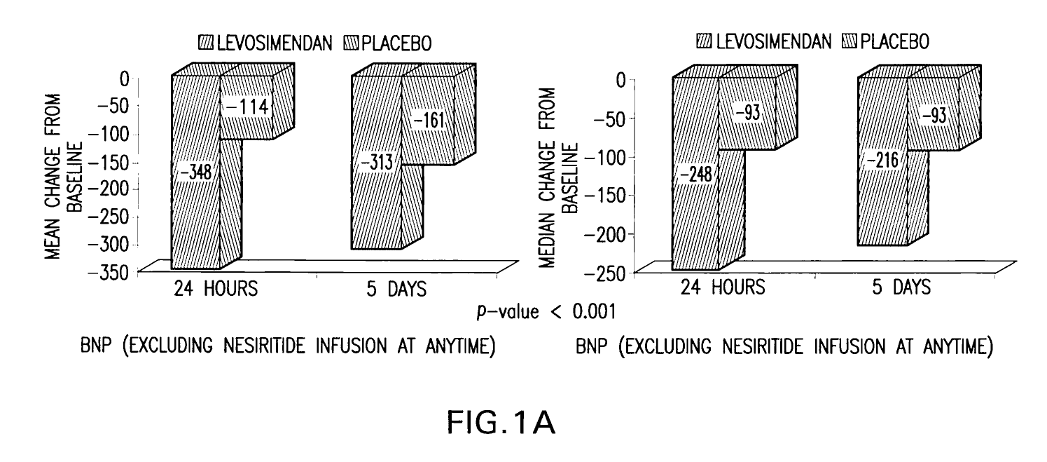

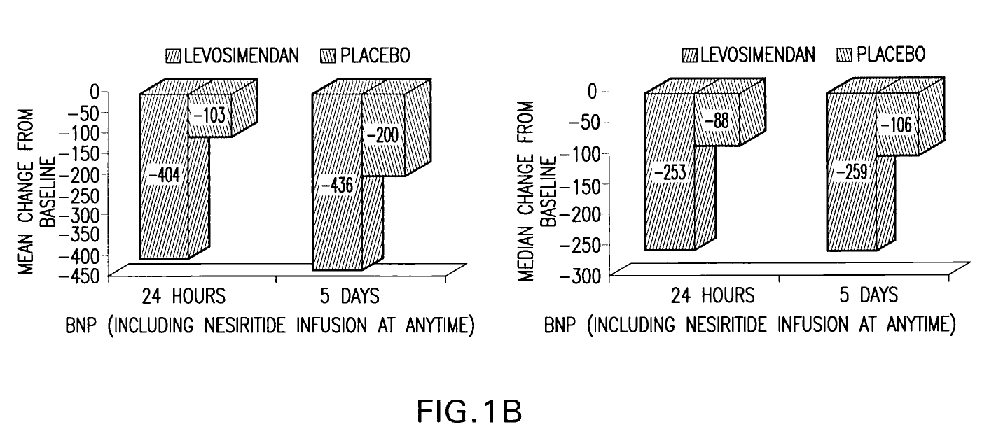

In the REVIVE II, serum BNP was serially followed during the study. The high baseline values (excluding patients administered nesiritide) were consistent with their severely impaired, decompensated heart failure status at enrollment (levosimendan=925 pg/mL and placebo=1001 pg/ml). The levosimendan treatment group experienced statistically significant decreases in BNP at 24 hours and 5 days (p=0.001 and p=0.001, respectively) compared to placebo on a background of standard-of-care therapy for each patient (FIG. 1A). These results are highly clinically relevant (FIG. 1B shows the results with patients administered nesiritide (BNP)). For example, several studies have demonstrated that elevated serum levels of B-type natriuretic peptide (BNP) are correlated with adverse outcomes in patients with heart failure (See Berkowitz, R., Rev. Cardiovasc. Med., 2004; 5 Suppl 4:S3-16). Furthermore, in a study of 72 patients hospitalized with decompensated heart failure, the mean level of BNP increased by 233 pg/mL in the patients who experienced an endpoint (death or readmission within 30 days), whereas the mean level of BNP decreased by 215 pg/mL in the patients without endpoints (See Cheng V., et al., J. Am. Coll. Cardiol. 2001; 37:386-391). The data indicated that changes in BNP levels during hospitalization are predictors for hospital readmissions and mortality. In another study by the same group, changes in BNP during hospitalization were correlated with changes in pulmonary capillary wedge pressure, and patients who died were found to have high final BNP levels (Kazanegra, R. et al., J. Card. Fail. 2001; 7:21-29).

The substantial and significantly greater reductions in BNP at both 24 hours and 5 days following levosimendan treatment (when compared to placebo) further supports the clinical evidence of an early and sustained clinical benefit of levosimendan. As discussed above, BNP declined during and after treatment in more patients treated with levosimendan than placebo. The effect of placebo on BNP remained consistent for 31 days. The effect of levosimendan on BNP diminished to a level similar to that of placebo at 31 days. Moreover, the exclusion of patients treated with nesiritide as mentioned above, did not change the magnitude of changes in BNP from baseline at any time point.

A recent study involving levosimendan and dobutamine (SURVIVE, see Example 2) showed similar reductions in BNP up to 31 days following levosimendan treatment. More specifically, the levosimendan treatment group, who were not administered nesiritide (except for one patient) experienced statistically significant decreases in BNP compared to dobutamine.

Patient Reported Responses

The Patient Global Assessment index captured the overall subject-reported response to therapy with a seven-category scale, ranging from markedly improved to markedly worsened. This index was independent of clinical events. The levosimendan treatment group experienced an overall trend in improvement in the Patient Global Assessment index over 5 days, with a statistically significant improvement at 24 hours and 5 days (p=0.026 and p=0.001, respectively) indicating that levosimendan produces rapid and durable clinical benefit to subjects. These data are displayed in Table 5, below.

TABLE 5
Patient Global Assessment

| | Levosimendan | | Placebo | |
|---|---|---|---|---|
| | (N = 299) | | (N = 301) | |
| Patient Global Assessment | N | % | N | % |
| Markedly improved | 19 | 6.4 | 22 | 7.3 |
| Moderately improved | 66 | 22.1 | 50 | 16.6 |
| Mildly improved | 120 | 40.1 | 102 | 33.9 |
| No change | 76 | 25.4 | 107 | 35.5 |
| Mildly worse | 5 | 1.7 | 5 | 1.7 |
| Moderately worse | 6 | 2.0 | 3 | 1.0 |
| Markedly worse | 7 | 2.3 | 12 | 4.0 |

| | |
|---|---|
| Patient Global Assessment 6 hours | p-value = 0.081 |

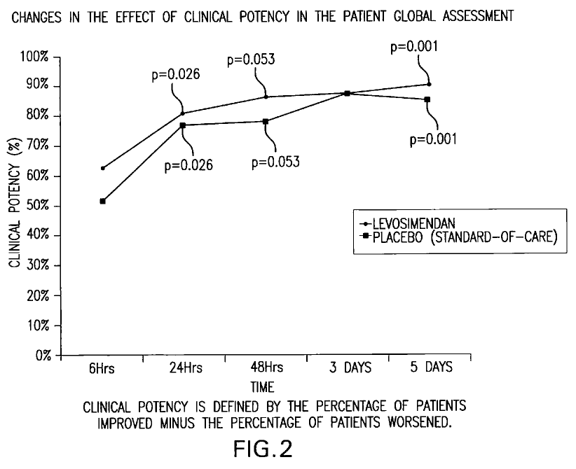

FIG. 2 shows comparison data in clinical potency (percentage improved minus percentage worsened) for Patient Global Assessment. The data reveal that levosimendan has significant and sustained improvement in clinically relevant parameters over placebo on a background of standard-of-care therapy.

Patient Dyspnea Assessment

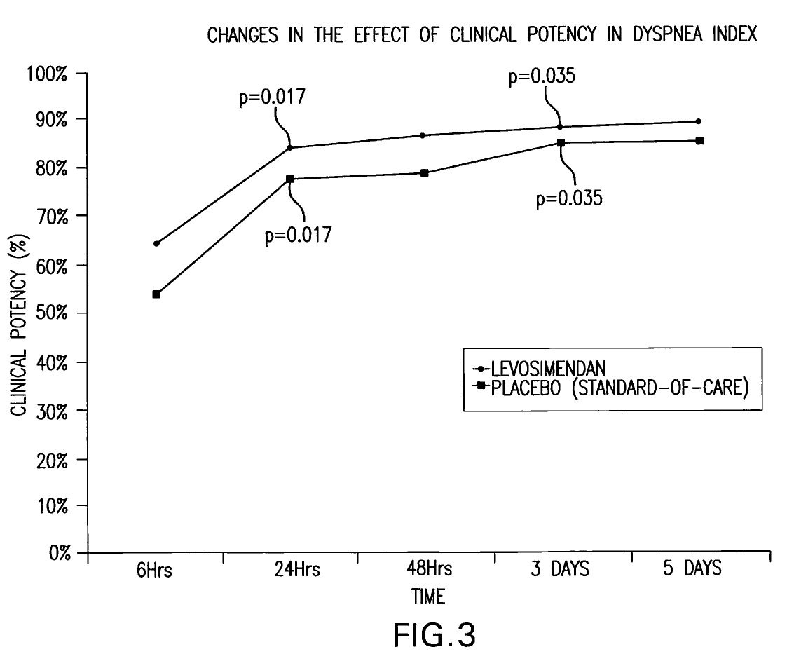

Patients' perception of their change in dyspnea status with treatment was also captured on a seven-category rank order scale (markedly improved, moderately improved, mildly improved, no change, mildly worse, moderately worse, markedly worse) at 6, 24 and 48 hours and Day 3 and Day 5. In order to standardize the assessment procedure, the patient was instructed to lie flat (such as in a bed (a single pillow is permitted)) to evaluate the current symptom severity at each and every assessment timepoint, if their condition allowed. If the patient was unable to lie flat, then the degree of elevation actually used was recorded in the patient's notes and all subsequent assessments were performed in the exactly the same position. A rapid positive response to levosimendan as assessed by the proportion of subjects demonstrating moderate or marked improvement was observed as displayed in Table 6, below. The data reveal that levosimendan has significant and sustained improvement in clinically relevant parameters over placebo on a background standard-of-care therapy consistent with the overall response noted with the Clinical Composite Endpoint and the Patient Global Assessment Index. These data are displayed in FIG. 3.

TABLE 6
Patient Dyspnea Assessment

| | Levosimendan | | Placebo | |
|---|---|---|---|---|
| | (N = 299) | | (N = 301) | |
| Patient Dyspnea Assessment | N | % | N | % |
| Markedly improved | 21 | 7.0 | 23 | 7.6 |
| Moderately improved | 70 | 23.4 | 53 | 17.6 |
| Mildly improved | 115 | 38.5 | 108 | 35.9 |
| No change | 77 | 25.8 | 94 | 31.2 |
| Mildly worse | 5 | 1.7 | 7 | 2.3 |
| Moderately worse | 3 | 1.0 | 4 | 1.3 |
| Markedly worse | 8 | 2.7 | 12 | 4.0 |

| | |
|---|---|
| Patient Global Assessment 6 hours | p-value = 0.078 |

Duration of Initial Hospitalization

The durations of patients' total hospitalization and intensive care unit (ICU) length-of-stay were evaluated during the study as depicted on Table 7, below. Results for both were consistent with the improved patient status in those receiving levosimendan.

TABLE 7
Duration of Initial Hospitalization (IH)

| | Levosimendan | | Placebo | | p-value |
|---|---|---|---|---|---|
| Duration of Initial Hospitalization | N = 299 | | N = 301 | | |
| Mean Duration of Initial Hospitalization | 7.07 | | 9.00 | | 0.001 |
| (days) | | | | | |
| Length of Stay-IH (Patients per period) | | | | | 0.012 |
| 1 to 5 days | 111 | 37.8% | 131 | 46.1% | |
| 6 to 10 days | 116 | 39.5% | 109 | 38.9% | |
| Over 10 days | 67 | 32.8% | 44 | 15.5% | |
| Total Patients in ICU (%) | N = 176 (58.9%) | | N = 177 (58.8%) | | 0.157 |
| Mean Duration of ICU Length | 5.4 | | 5.9 | | 0.210 |
| of Stay (days) | | | | | |
| Number of re-hospitalizations within | | | | | |
| 31 days of initial discharge | | | | | |
| Total patients re-hospitalized - 31 | 72 | | 80 | | 0.806 |
| days | | | | | |
| One event n/N patients (%) | 62/72 | 86.1% | 69/80 | 86.2% | |
| Two events n/N patients (%) | 8/72 | 11.1% | 10/80 | 12.5% | |
| Three events n/N patients (%) | 2/72 | 2.7% | 1/80 | 1.2% | |

Mean Days alive out of hospital

| | | | |
|---|---|---|---|
| Up to 14 days | 6.7 | 6.2 | 0.176 |
| Up to 31 days | 20 | 20 | 0.850 |
| Up to 90 days | 67 | 69 | 0.177 |

Time to Death or Worsening of Heart Failure

Although not statistically significant, there was a trend in favor of the levosimendan treatment group in time to death or worsening heart failure in the first 31 days measured in mean days (9.4 versus 6.7 days, p-value=0.102) as displayed in Table 8, below.

TABLE 8
Time to Death or Worsening of Heart Failure

| Death or worsening of | Levosimendan | | Placebo | | |
|---|---|---|---|---|---|
| heart failure | N | % | N | % | p-value |
| Yes | 100 | 33.4% | 118 | 39.2% | 0.102 |
| Time to death or WHF | | | | | |
| (Days) | | | | | |

| | | |
|---|---|---|
| Mean (SD) | 9.4 (9.4) | 6.7 (8.5) |
| Median | 5 | 3 |

p-value from Cochran-Mantel-Haenszel test from stratified model

New York Heart Association (NYHA) Classification

Physicians evaluated patients' NYHA classification, an evaluation of patients' functional status, at day 5. The overall results are illustrated in Table 9, below.

TABLE 9
NYHA Classification at Day 5

| | |
|---|---|
| Levosimendan | Placebo |

| NYHA class | N | % | N | % | p-value |
|---|---|---|---|---|---|
| Class I | 5 | 1.7% | 4 | 1.3% | 0.196 |
| Class II | 62 | 20.7% | 49 | 16.3% | |
| Class IIIa | 133 | 44.5% | 14 | 46.5% | |
| Class IIIb/IV | 99 | 33.1% | 108 | 135.9% | |

p-value from Cochran-Mantel-Haenszel test from stratified model

Renal Function

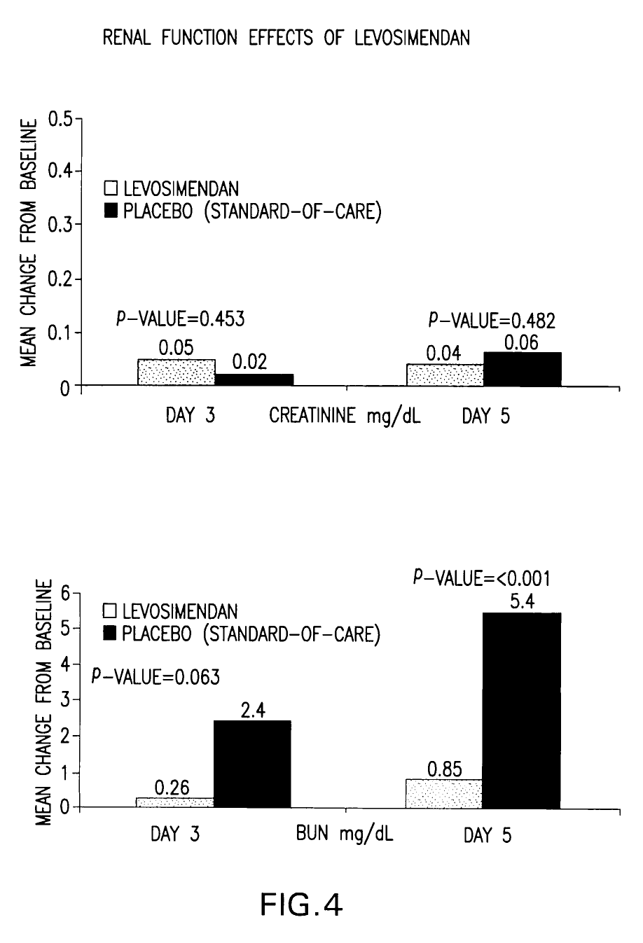

The levosimendan treatment group was comparable to placebo in mean change in serum creatinine at day 3 and 5 (FIG. 4). In addition, the levosimendan treatment group had a smaller mean change in blood urea nitrogen (BUN) at day 3 and 5 compared to placebo on a background of standard of care (FIG. 4). These data were confirmed by a more stringent analysis of renal compromise, where levosimendan and placebo therapy again were not different in terms of the proportions of patients experiencing an increase of at least 0.5 mg/dL serum creatinine over baseline at day 31 (8.5% versus 8%; p-value=0.852).

This may have clinical relevance as several studies conducted within the last 5 years indicate that increased serum creatinine levels in patients hospitalized for heart failure are associated with adverse outcomes, including prolonged hospitalization and increased mortality. A study of 1681 Medicare beneficiaries with heart failure showed that worsened renal function (defined as >0.3 mg/dL increase in serum creatinine) during hospitalization was associated with prolonged lengths of hospitalization, higher in-hospital costs, increased in-hospital mortality, and an increased likelihood of readmission (Krumholz H M, et al., Am. J. Cardiol., 2000; 85-1110-1113). These results were confirmed in a more-diverse population of 1004 patients with heart failure, demonstrating that serum creatinine increases >0.3 mg/dL, during hospitalization were associated with increased complications, length of hospital stay, and in-hospital mortality (Forman D E, et al., J. Am. Coll. Cardiol. 2004; 4, 3:61-67).

Gottlieb et al conducted a retrospective review of 1002 chronic heart failure patients hospitalized for heart failure and concluded that any detectable increase in serum creatinine levels during hospitalization for heart failure was associated with prolonged hospitalization and increased mortality (See Gottlieb et al., J. Card. Fail, 2002; 8:135-141). Similar results were obtained in a prospective analysis of 412 patients hospitalized for heart failure (Smith G L, et al., J. Card. Fail., 2003; 9:13-25). In this study, the risk of death rose with higher creatinine elevations (adjusted hazard ratios 1.19, 1.67, 1.91, and 2.90 for elevations of ≧0.2 mg/dL, ≧0.3 mg/dL, ≧0.4 mg/dL, and ≧0.5 mg/dL). A post-hoc analysis of data from the VMAC (nesiritide pivotal study) trial indicated that worsening renal function (defined as >0.5 mg/dL increase in serum creatinine) was associated with a significantly longer length of hospital stay (8 versus 6 days, p-value<0.001) and a significantly increased 6-month mortality (risk ratio 1.61, 95% confidence interval [CI] 1.09-2.37, p-value=0.02) (See Akhter M W, et al., Am J. Cardiol., 2004; 94:957-960).

An analysis by Fonarrow et al of the Acute Decompensated Heart Failure National Registry (ADHERE) revealed that high admission levels of BUN (≧43 mg/dL or 15.35 mmol/L) and creatinine (≧2.75 mg/dl or 243.1 micromol/L) were two of the three best predictors of mortality of 39 variables studied (Fonarrow G C, et al., JAMA, 2005; 293:572-580).

Safety

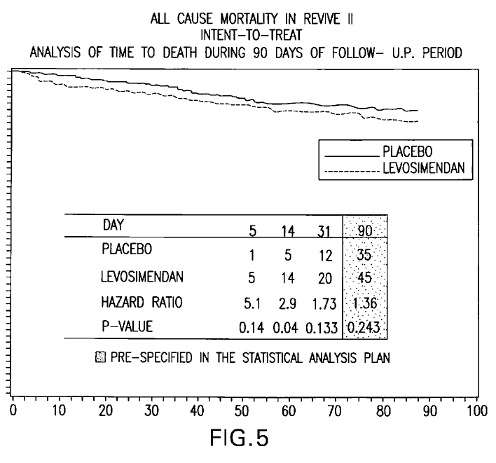

Mortality—At the pre-specified time point of 90 days, there was an increase in mortality that was not statistically significant in the levosimendan treated patients compared to placebo on a background therapy of standard-of-care, 45 versus 35 deaths. Additional analyses of mortality, at day 5 due to the span of the primary endpoint, and day 14 due to the duration of the exposure to the long acting metabolite were conducted, and the results are also shown in FIG. 5.

The excess deaths in the levosimendan treatment group occur early in the course of therapy as seen at day 5, representing course of initial therapy and hospitalization, and day 14, representing exposure to the long-acting metabolite of levosimendan patient.

Adverse Events

The overall safety results were generally similar and largely confirmed results from previous trials. Adverse events were more frequently reported in the levosimendan group (92.8% vs. 90.5%). Serious adverse events were more frequently reported in the levosimendan group (36.5% vs. 33.3%).

The adverse events of special interest were classified into six groups in order to assess the safety of the product from a cardiovascular and renal perspective in line with the expectation of the adverse events profile of products used for acute decompensated heart failure. The classifications of these groups are as follows:
Group 1: Atrial fibrillation, atrial flutter, supraventricular tachycardia, supraventricular extrasystoles and atrial tachycardia.
Group 2: Ventricular extrasystoles, ventricular fibrillation, ventricular tachycardia, Extrasystoles and torsade de pointes
Group 3: Group 1 and 2 together
Group 4: Acute coronary syndrome, acute myocardial infarction, angina pectoris, angina unstable, cardiac arrest, coronary artery disease, ischemic cardiomyopathy and myocardial infarction
Group 5: Hypotension
Group 6: Acute pre-renal failure, anuria, azotemia, azotemia, oliguria, renal failure, acute renal failure, renal impairment, blood creatinine increased, blood urea increased and urine output decreased.

The most common adverse events of special interest reported through day 31 in both groups were arrhythmias and hypotension, all of which occurred at a higher frequency in the levosimendan-treated subjects. The differences in the rates of these adverse events between groups do not appear to differ over time (data at day 5 and 14 not shown). The coronary and renal adverse events were similar in both treatment groups as depicted in Table 10, below.

TABLE 10
Summary of Patients who had AEs
Adverse Event - Day 31
Revive II - ITT Population

AE Up to Day 31

| | |
|---|---|
| Levosimendan | Placebo |
| (N = 299) | (N = 301) |

| | N | % | N | % | p-value^{$} |
|---|---|---|---|---|---|
| Group 1 | 42 | 14.0 | 16 | 5.3 | <0.001*** |
| Group 2 | 87 | 29.1 | 57 | 18.9 | 0.004** |
| Group 3 | 27 | 9.0 | 28 | 9.3 | NS |
| Group 4 | 151 | 50.5 | 109 | 36.2 | <0.001*** |
| Group 5 | 51 | 17.1 | 51 | 16.9 | NS |

^{$}p-values are from Fisher's exact test
*p-values ≦ 0.05
**p-values ≦ 0.01
***p-values ≦ 0.001
NS = not significant
Group 1 Atrial fibrillation, atrial flutter, atrial tachycardia, supraventricular extrasystoles, and supraventricular tachycardia.
Group 2 Extrasystoles, torsade de pointes, ventricular arrhythmia, ventricular extrasystoles, ventricular fibrillation, ventricular flutter, ventricular tachycardia.
Group 3 Acute coronary syndrome, acute myocardial infarction, angina pectoris, angina unstable, cardiac arrest, cardio-respiratory arrest, coronary artery disease, coronary artery stenosis, ischemic cardiomyopathy, myocardial infarction, myocardial ischemia, post-infarction angina, sinus arrest, sub-endocardial ischemia.
Group 4 Blood pressure decreased, hypotension.
Group 5 Acute pre-renal failure, acute renal failure, anuria, azotemia, blood creatinine increased, blood urea increased, oliguria, renal failure, renal impairment, urine output decreased.

Additional Analysis of Survival Data

The plausibility of an increased number of mortality events in the levosimendan treatment group, while unexpected, deserved examination within the context of the study.

Although these analyses were post-hoc and retrospective, they utilized predefined randomized baseline values for SBP<100 mmHg. These evaluations examined subsets of the randomized population, which were not pre-specified.

Systolic Blood Pressure

Based on the literature identifying systolic blood pressure along with other markers as being useful in identifying hospitalized acutely decompensated heart failure patients that were at risk for mortality (See Fonarrow G C, et al., JAMA, 2005; 293:572-580 and Felker G M, et al., J. Card Fail. 2004; 10:460-466), the inventors hypothesized that patients with lower blood pressures at baseline may be at particular risk of a poor outcome.

The eligibility criteria for the REVIVE II study excluded patients who had a baseline systolic blood pressure (SBP)≦90 mm Hg thus setting a lower boundary for this analysis. The inventors investigated the outcome of mortality in the subgroup of patients with a SBP<100 mm Hg at baseline (See Table 11). In REVIVE II, this population was relatively small, constituting 20% of total the intent-to-treat patient set.

The data in Table 11 below suggests that patients who had a baseline SBP<100 mm Hg and received levosimendan were at a higher risk of mortality compared to the other three subgroups. This risk occurred within the three time periods examined.

TABLE 11
Mortality Events
Baseline SBP < 100 mmHg vs Baseline SBP > 100 mmHg

| | 14 Days | | 31 Days | | 90 Days | |
|---|---|---|---|---|---|---|
| | N | % | N | % | N | % |
| All Patients | | | | | | |
| 1. Levosimendan | 14/299 | 4.7 | 20/299 | 6.7 | 45/299 | 15% |
| (N = 299) | | | | | | |
| 2. Placebo | 5/301 | 1.7 | 12/301 | 4.0 | 35/301 | 11.6% |
| (N = 301) | | | | | | |
| Levosimendan | | | | | | |
| (N = 297) | | | | | | |
| 3. <100 mmHg | 8/56 | 14.3 | 10/56 | 17.9 | 17/56 | 30.3 |
| 4. ≧100 mmHg | 6/243 | 2.5 | 10/243 | 4.1 | 28/243 | 11.5 |
| Placebo (N = 299) | | | | | | |
| 5. <100 mmHg | 1/64 | 1.6 | 2/64 | 3.1 | 10/64 | 15.6 |
| 6. ≧100 mmHg | 4/237 | 1.7 | 10/237 | 4.2 | 24/237 | 10.5 |

The risk of death in the levosimendan SBP≧100 mm Hg group is similar to the risk of death in the overall placebo group (lines 4 and 2 of Table 11). The relative risks along with confidence intervals for within treatment group comparisons are listed in Table 12, below.

TABLE 12
Relative Risk Stratified by Baseline Systolic Blood Pressure

| | 14 Days | 31 Days | 90 Days |
|---|---|---|---|
| | RR (CI) | RR (CI) | RR (CI) |
| Levosimendan | | | |
| 1. <100 mmHg compared | 5.9 (2.1-16.2) | 4.4 (1.9-99.9) | 2.7 (1.6-4.5) |
| to | | | |
| 2. ≧100 mmHg | | | |
| Placebo | | | |
| 3. <100 mmHg compared | 0.9 (0.1-8.1) | 0.7 (0.2-3.3) | 1.5 (0.8-3.0) |
| to | | | |
| 4. ≧100 mmHg | | | |

Concurrent Therapy

The inventors also hypothesized that that patients receiving concomitant vasoactive therapy (i.e., inotropic drugs and vasodilators) were at greater risk for poor outcomes. This hypothesis was also investigated.

Dividing the above sub-populations by concomitant medication use and focusing on drugs that have vasodilatory properties and a propensity to affect blood pressure, a working hypothesis was that if levosimendan patients with lower SBP are vulnerable, this vulnerability would be amplified if the group were to be exposed to agents that have the potential to decrease blood pressure.

Nesiritide, a vasodilator that functions via a nitric oxide pathway, is currently approved for use in the United States in patients with acutely decompensated heart failure. In this study, nesiritide could be utilized as part of the standard-of-care treatment regimen.

When nesiritide is administered within the first 14 day to patients treated with levosimendan, the rate of death is greatest in the baseline SBP<100 mm Hg group (Table 13, line 5). Additionally, the mortality rate is also moderately elevated in the levosimendan SBP>100 mm Hg group (Table 13 line 6) relative to the corresponding placebo group (Table 13 line 8 below).

TABLE 13
Mortality Rate by Baseline Systolic Blood Pressure
Strata Without Nesiritide Use

| 14 Days | | 31 Days | | 90 Days | |
|---|---|---|---|---|---|
| N | % | N | % | N | % |

Without Nesiritide

| | | | | | | |
|---|---|---|---|---|---|---|
| Levosimendan | | | | | | |
| (N = 297) | | | | | | |
| 1. <100 mmHg | 3/39 | 7.7 | 4/39 | 10.3 | 7/39 | 18.0 |
| 2. ≧100 mmHg | 3/195 | 1.5 | 4/195 | 2.1 | 19/195 | 9.7 |
| Placebo (N = 299) | | | | | | |
| 3. <100 mmHg | 1/51 | 2.0 | 2/51 | 3.9 | 9/51 | 17.7 |
| 4. ≧100 mmHg | 2/174 | 1.2 | 6/174 | 3.5 | 16/174 | 9.2 |

With Nesiritide

| | | | | | | |
|---|---|---|---|---|---|---|
| Levosimendan | | | | | | |
| (N = 297) | | | | | | |
| 5. <100 mmHg | 5/16 | 31.3 | 6/16 | 37.5 | 10/16 | 62.5 |
| 6. ≧100 mmHg | 3/47 | 6.4 | 6/47 | 12.8 | 9/47 | 19.2 |
| Placebo (N = 299) | | | | | | |
| 7. <100 mmHg | 0/13 | 0 | 0/13 | 0 | 1/13 | 7.7 |
| 8. ≧100 mmHg | 2/61 | 3.3 | 4/61 | 6.6 | 8.61 | 13.1 |

The corresponding relative risk among the above groups is listed in Table 14, below. As a reference lines 1 and 2 provide the relative risks from Table 13 for each overall treatment group.

TABLE 14
Relative Risk Stratified by Baseline Systolic Blood Pressure and Nesiritide Use

| | 14 Days | 31 Days | 90 Days |
|---|---|---|---|
| | RR (CI) | RR (CI) | RR (CI) |
| Levosimendan (N = 301) | | | |
| 1. <100 mmHg compared to ≧100 mmHg | 5.9 (2.1-16.2) | 4.4 (1.9-10.1) | 2.7 (1.6-4.5) |
| Placebo (N = 299) | | | |
| 2. <100 mmHg compared to ≧100 mmHg | 0.9 (0.1-8.1) | 0.7 (0.2-2.3) | 1.5 (0.8-3.0) |
| Levosimendan < 100 mmHg | | | |
| 3. Plus Nesiritide compared to without | 4.1 (1.1-15.0) | 3.7 (1.2-11.2) | 3.5 (1.6-7.5) |
| Nesiritide | | | |
| Placebo < 100 mmHg | | | |
| 4. Plus Nesiritide compared to without | 1.2 (0.1-28.7) | 0.7 (0.0-14.6) | 0.4 (0.1-3.1) |
| Nesiritide | | | |
| Levosimendan > 100 mmHg | | | |
| 5. Plus Nesiritide compared to without | 4.1 (0.9-2.0) | 6.2 (1.8-21.2) | 2.0 (1.0-4.1) |
| Nesiritide | | | |
| Placebo > 100 mmHg | | | |
| 6. Plus Nesiritide compared to without | 2.9 (0.4-19.8) | 1.9 (0.6-6.5) | 1.4 (0.6-3.2) |
| Nesiritide | | | |

Similar analyses were performed for other vasoactive agents, which are commonly used in heart failure, such as dobutamine and milrinone, which can produce notable blood pressure effects (Table 14-Table 18). The risk for an event when these medications are used seems to be greatest in patients receiving levosimendan with a SBP<100 mm Hg, consistent with the previous observation for nesiritide.

In contrast to nesiritide, the combination of either dobutamine or milrinone with levosimendan in patients with a baseline SBP≧100 mm Hg performed similarly or better in terms of mortality relative to the comparable placebo groups. While not wishing to be bound by any theory, one factor that may explain this difference between the relative effects of nesiritide compared to the other vasoactive agents is that nesiritide is solely a vasodilator. Whereas the other two agents produce both vasodilatory and positive inotropic activities, which may minimize the propensity of a marked blood pressure effect.

TABLE 15
Mortality Rate by Baseline Blood Pressure Strata
With or Without Dobutamine Use

| 14 Days | | 31 Days | | 90 Days | |
|---|---|---|---|---|---|
| N | % | N | % | N | % |

Without Dobutamine

| | | | | | | |
|---|---|---|---|---|---|---|
| Levosimendan | | | | | | |
| 1. <100 mmHg | 1/39 | 2.6 | 3/39 | 7.7 | 9/39 | 23.1 |
| 2. ≧100 mmHg | 3/199 | 1.5 | 7/199 | 3.5 | 21/199 | 10.6 |
| Placebo | | | | | | |
| 3. <100 mmHg | 1/41 | 2.4 | 1/41 | 2.4 | 4/41 | 9.8 |
| 4. ≧100 mmHg | 2/197 | 1.0 | 3/197 | 1.5 | 13/197 | 6.6 |

With Dobutamine

| | | | | | | |
|---|---|---|---|---|---|---|
| Levosimendan | | | | | | |
| 5. <100 mmHg | 7/16 | 43.8 | 7/16 | 43.3 | 8/16 | 50.0 |
| 6. ≧100 mmHg | 3/43 | 7.0 | 3/43 | 7.0 | 7/43 | 16.3 |
| Placebo | | | | | | |
| 7. <100 mmHg | 0/23 | 0 | 1/23 | 4.4 | 6/23 | 26.1 |
| 8. ≧100 mmHg | 2/38 | 5.3 | 7/38 | 18.4 | 11/38 | 29.0 |

TABLE 16
Relative Risk Stratified by Baseline Systolic Blood Pressure and Dobutamine Use

| | 14 Days | 31 Days | 90 Days |
|---|---|---|---|
| | RR (CI) | RR (CI) | RR (CI) |
| Levosimendan (N = 301) | | | |
| 1. <100 mmHg compared to ≧100 mmHg | 5.9 (2.1-16.2) | 4.4 (1.9-10.1) | 2.7 (1.6-4.5) |
| Placebo (N = 299) | | | |
| 2. <100 mmHg compared to ≧100 mmHg | 0.9 (0.1-8.1) | 0.7 (0.2-2.3) | 1.5 (0.8-3.0) |
| Levosimendan < 100 mmHg | | | |
| 3. With Dobutamine compared to | 17.1 (2.3-12.8) | 5.7 (1.7-19.3) | 2.2 (1.0-4.6) |
| without Dobutamine | | | |
| Placebo < 100 mmHg | | | |
| 4. With Dobutamine compared to | 0.6 (0.0-13.8) | 1.8 (0.1-27.2) | 2.7 (0.8-8.5) |
| without Dobutamine | | | |
| Levosimendan ≧ 100 mmHg | | | |
| 5. With Dobutamine compared to | 4.6 (1.0-22.2) | 2.0 (0.5-7.4) | 1.5 (0.7-3.4) |
| without Dobutamine | | | |
| Placebo ≧ 100 mmHg | | | |
| 6. With Dobutamine compared to | 5.2 (0.8-35.7) | 12.1 (3.3-44.6) | 4.4 (2.1-9.0) |
| without Dobutamine | | | |

TABLE 17
Mortality Rate by Blood Pressure Strata With or Without Milrinone Use

| 14 Days | | 31 Days | | 90 Days | |
|---|---|---|---|---|---|
| N | % | N | % | N | % |

Without Milrinone

| | | | | | | |
|---|---|---|---|---|---|---|
| Levosimendan | | | | | | |
| (N = 297) | | | | | | |
| 1. <100 mmHg | 5/47 | 10.6 | 7/47 | 14.9 | 14/47 | 29.8 |
| 2. ≧100 mmHg | 5/229 | 2.2 | 9/229 | 3.9 | 26/229 | 11.4 |
| Placebo (N = 299) | | | | | | |
| 3. <100 mmHg | 1/52 | 1.9 | 2.52 | 3.9 | 10/52 | 19.2 |
| 4. ≧100 mmHg | 3/221 | 1.4 | 8/221 | 3.6 | 21/221 | 9.5 |

With Milrinone

| | | | | | | |
|---|---|---|---|---|---|---|
| Levosimendan | | | | | | |
| (N = 297) | | | | | | |
| 5. <100 mmHg | 3/8 | 37.5 | 3/8 | 37.5 | 3/8 | 37.5 |
| 6. ≧100 mmHg | 1/13 | 7.7 | 1/13 | 7.7 | 2/13 | 15.4 |
| Placebo (N = 299) | | | | | | |
| 7. <100 mmHg | 0/12 | 0 | 0/12 | 0 | 0/12 | 0 |
| 8. ≧100 mmHg | 1/14 | 7.1 | 2/14 | 14.3 | 3/14 | 21.4 |

TABLE 18
Relative Risk Stratified by Baseline Systolic Blood Pressure and Milrinone Use

| | 14 Days | 31 Days | 90 Days |
|---|---|---|---|
| | RR (CI) | RR (CI) | RR (CI) |
| Levosimendan (N = 301) | | | |
| <100 mmHg compared to ≧100 mmHg | 5.9 (2.1-16.2) | 4.4 (1.9-10.1) | 2.7 (1.6-4.5) |
| Placebo (N = 299) | | | |
| <100 mmHg compared to ≧100 mmHg | 0.9 (0.1-8.1) | 0.7 (0.2-2.3) | 1.5 (0.8-3.0) |
| Levosimendan < 100 mmHg | | | |
| With Milrinone compared to without | 3.5 (1.0-11.9) | 2.5 (0.8-7.8) | 1.3 (0.5-3.4) |
| Milrinone | | | |
| Placebo < 100 mmHg | | | |
| With Milrinone compared to without | 1.4 (0.1-3.4) | 0.8 (0.0-16.0) | 0.2 (0.0-3.1) |
| Milrinone | | | |
| Levosimendan ≧ 100 mmHg | | | |
| With Milrinone compared to without | 3.5 (0.4-28.0) | 2.0 (0.3-14.3) | 1.4 (0.4-5.1) |
| Milrinone | | | |
| Placebo ≧ 100 mmHg | | | |
| With Milrinone compared to without | 5.3 (0.6-47.4) | 3.9 (0.9-16.9) | 2.3 (0.8-6.7) |
| Milrinone | | | |

Efficacy Analysis of Patients with SBP>100 mmHg

In order to discern the overall impact of the retrospective analysis in identifying the subset of patients with SBP<100 mm Hg, the primary endpoint analysis was repeated without the inclusion of this patient cohort.

In this post-hoc analysis of the clinical composite primary endpoint the removal of these patients, who constituted 20% (121/600) of the total enrollment, has little impact on the assessment of the overall efficacy of levosimendan. In the remaining 479 patients, the primary endpoint demonstrated a treatment benefit for levosimendan over that of placebo which was nearly identical to that found in the original ITT population of 600 patients (Table 19).

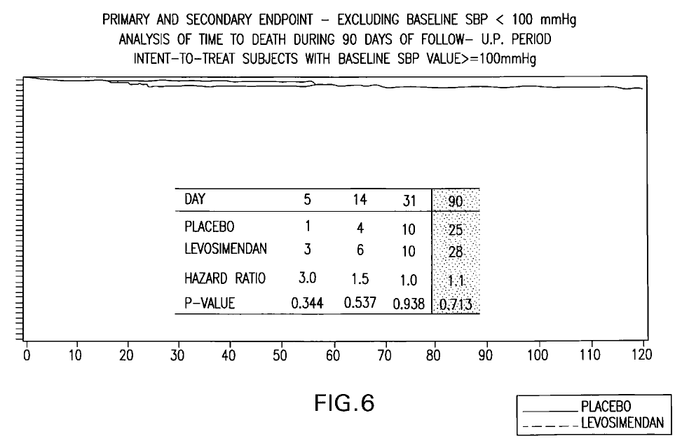

Corresponding mortality data excluding the SBP<100 mm Hg sub-group is depicted in FIG. 6 demonstrating that the event rates are similar between groups.

TABLE 19
Primary Endpoint Analysis excluding SBP < 100 mm HG

| | | |
|---|---|---|
| Levosimendan | Placebo | Overall |
| N = 299 | N = 301 | p-value |

| | | | | | |
|---|---|---|---|---|---|
| Improved | 51 | 20.9% | 35 | 14.7% | 0.0187 |
| Unchanged | 148 | 60.9% | 142 | 59.9% | |
| Worse | 44 | 18.1% | 60 | 25.3% | |

p-value from Cochran-Mantel-Haenszel test from stratified model

### EXAMPLE 2

### The SURVIVE Study

The purpose of this study was to examine the survival of patients with acute heart failure when administered intravenous levosimendan versus intravenous dobutamine. This study was a multi-center study and a total of 94 sites were initiated. These sites were: Austria 4, Finland 6, France 31, Germany 14, Israel 7, Latvia 6, Poland 8, Russia 9, United Kingdom 9; of these, 75 sites randomized at least one patient.

More specifically, the primary objective of the study was to compare the efficacy of levosimendan and dobutamine on “all-cause mortality” in the 180 days following randomization. The secondary objectives of the study were to evaluate the efficacy of levosimendan compared to dobutamine on:
1. All-cause mortality during the 31 days following randomization.
2. Mean change in plasma BNP concentration from baseline to 24 hours after start of the study drug infusion.
3. Number of days alive out of hospital (DAOH) during the 180 days following randomization.
4. Patient's Assessment of Dyspnea at 24 hours following randomization.
5. Patient's Global Assessment at 24 hours following randomization. The Patient's Global Assessment used in the SURVIVE study was the same Patient's Global Assessment study used in the REVIVE I and REVIVE II Studies which is described in Example 1.
6. Cardiovascular mortality during the 180 days following randomization.

Mean change in plasma BNP concentration from baseline to 24 hours was not specified as a secondary objective in the protocol, but was added as a secondary endpoint in the Statistical Analysis Plan (hereinafter “SAP”) prior to blind break.

### Methodology:

This was a randomized, double-blind, double-dummy, active-controlled, parallel-group, multicenter study. Patients received two simultaneous intravenous (hereinafter “IV”) infusions, levosimendan or placebo for levosimendan, with a duration of 24 hours, and dobutamine or placebo for dobutamine, with duration according to clinical judgment, but minimally for 24 hours.

The study consisted of a screening period (from informed consent to baseline) (hereinafter “Screening Period”), a treatment period (from baseline to completion or termination of study drug infusions) (hereinafter “Treatment Period”), and a follow-up period (from completion or termination of study drug infusions to 180 days) (hereinafter “Treatment Period”).

During the Follow-up Period, the randomized study drug assignment was maintained and re-administered in a blinded fashion when clinically justified. In such case, every effort to administer the same study drug as during the initial study drug administration was to be made. Study drug could be re-administered only in the same study center in which the initial administration of the study drug was performed. The number of re-administrations was not limited. If the initial study drug infusion was prematurely discontinued due to dose-limiting event (hereinafter “DLE”) or adverse event, then re-administration was not allowed.

Pre-scheduled follow-up visits took place at 31 (+5 days), 90 (+10 days), and 180 (+10 days) days from randomization. Additionally, the patient was contacted by telephone by the study site personnel on Days 60 (±5 days), 120 (±5 days), and 150 (±5 days).

Mortality was recorded to day 180 following randomization. Hospitalization was recorded from randomization to day 180 or death. The patient completed a dyspnea evaluation and global assessment at 24 hours following randomization. New York Heart Association (hereinafter “NYHA”) class was determined at baseline, at 24, 48, 72, 96, and 120 hours, and at 31, 90, and 180 days following randomization. B-type natriuretic peptide (hereinafter “BNP”) and N-terminal proBNP (hereinafter “NT-proBNP”) were determined at baseline and at 24, 72, and 120 hours.

Safety parameters such as adverse events, vital signs, 12-lead electrocardiograms (hereinafter “ECGs”), laboratory tests, and concomitant medications were monitored frequently throughout the Treatment and Follow-up Periods. Adverse events were collected for 31 days following the initial administration as well as all re-administration of blinded study drug.

### Number of Subjects (Planned and Analyzed):

Planned: Enrollment was to continue until minimally 330 deaths were anticipated to occur through the 180-day follow-up for all randomized patients (a total of approximately 1300 randomized patients, 650 per group).

Analyzed: 1327 (664=levosimendan, 663=dobutamine) with total 358 deaths (173=levosimendan deaths, 185=dobutamine deaths) through 180 days.

Diagnosis and Main Criteria for Inclusion:

Inclusion Criteria:

1. Written, signed, and dated informed consent.

2. Male and female patients over 18 years of age. Females of childbearing potential must have had a negative pregnancy test and refrained from breastfeeding. Women who were postmenopausal (2 years since last menstrual cycle), surgically sterilized or who had undergone a hysterectomy were considered not to be of childbearing potential.

3. Hospitalized patients with acutely decompensated heart failure

4. Left ventricular ejection fraction less than or equal to 30% as assessed using echocardiography, radionuclide ventriculography or contrast angiography within previous 12 months (prior to inclusion).

5. Clinical need for IV inotropic support as evidenced by insufficient response to IV diuretics and/or vasodilators (nitroglycerin, nitroprusside) and at least one of the following at screening:

oliguria (mean urine output<30 mL/h for at least 6 hours) and not a result of hypovolemia;

dyspnea at rest or mechanical ventilation for heart failure;

hemodynamic impairment in those patients with Swan-Ganz catheter inserted (pulmonary capillary wedge pressure [PCWP]≧18 mmHg and/or cardiac index≦2.2 L/min/m²).

### Exclusion Criteria:

1. Severe obstruction of ventricular outflow tracts such as hemodynamically significant uncorrected primary valve disease and restrictive or hypertrophic cardiomyopathy or impaired ventricular filling such as restrictive cardiomyopathy.
2. Weight≧160 kg.
3. Cardiac surgery within 30 days before screening.
4. Stroke within 3 months before screening.
5. Systolic blood pressure persistently less than 85 mmHg at screening or at baseline.
6. Heart rate persistently 130 bpm or greater at screening or at baseline.
7. Serum potassium less than 3.5 mmol/L at screening.
8. Administration of any inotropic agent (such as, dobutamine, milrinone, amrinone, enoximone, epinephrine, norepinephrine) except digitalis or dopamine (with dose of less than or equal than 2 μg/kg/min) during the current hospitalization
9. Hypersensitivity to levosimendan or dobutamine or any of their excipients.
10. A history of Torsade de Pointes.
11. Severe renal insufficiency (serum creatinine>450 μmol/L [5.0 mg/dL]) or on dialysis.
12. Significant hepatic impairment at discretion of the investigator
13. Acute bleeding,
14. Severe anemia (hemoglobin<8 g/dL) at screening.
15. Septicemia or septic shock.
16. Other serious diseases limiting life expectancy considerably (e.g., end-stage cancer).
17. Participation in a clinical trial with any experimental treatment within 30 days prior to screening or previous participation in the present study.
18. Administration of levosimendan within 30 days prior to screening.

### Test Product, Dose/Strength/Concentration, Mode of Administration:

Levosimendan was supplied as a concentrated solution (2.5 mg/mL). It was administered as an intravenous infusion (50 μg/mL in 5% glucose) through either a central or peripheral line according to the following schedule:

1. A total loading dose of 12 μg/kg administered over, 10 minutes,

2. A continuous infusion of 0.1 μg/kg/min for the following 50 minutes.

3. If well tolerated, the infusion rate was increased to 0.2 μg/kg/min for a further 23 hours.

4. If the higher dose was not well tolerated it was reduced to 0.1 μg/kg/min and then further to 0.05 μg/kg/min, as required.

5. If the patient did not tolerate this lowest dose, the medication was permanently discontinued.

6. The study drug infusion was stopped 24 hours after it was first started unless already discontinued for a dose-limiting event.

Reference Therapy, Dose/Strength/Concentration and Mode of Administration:

Dobutamine was supplied as a concentrated solution (12.5 mg/mL). It was administered at infusion rates and duration of treatment decided by the treating investigator, but the minimum infusion rate should have been at least 5 μg/kg/minute and the infusion duration should have been ≧24 hours
1. Treatment was started at an infusion rate of 5 μg/kg/min.
2. The dose was increased to achieve clinical goals, up to a dose of 40 μg/kg/min.
3. The dose was not reduced to below 5 μg/kg/min, unless for documented intolerance.
4. The infusion was maintained for as long as the physician deemed clinically appropriate, but minimally 24 hours.
5. The infusion was tapered off slowly.

### A calibrated infusion pump was used to deliver the infusions.

### Duration of Treatment:

24 hours (levosimendan); ≧24 hours (dobutamine).

### Re-Administration Protocol:

If the test drug was to be re-administered during this study, the following protocol was used. On re-administration Day 0 (at 0 hours), the following evaluations were recorded: reason(s) for re-administration; vital signs (HR and BP); 12-lead ECG; eligibility for re-administration (exclusion criteria 5, 6, 7, 9, 10, 11, 13, 14, 15); adverse events inquiry; and concomitant treatments. The levosimendan/placebo and dobutamine/placebo infusions were administered in accordance with the dosing instructions. If the levosimendan/placebo re-administration was within 7 days after starting the previous study drug infusion, the dose of levosimendan/placebo infusion was to be reduced.

At 6 hours (±30 minutes) vital signs (HR and BP) were recorded. At 24 hours (±1 hour), levosimendan/placebo infusion was stopped; the dobutamine/placebo infusion may have continued or may have been stopped gradually at any time between 24-48 hours at the investigator's discretion. In addition, the following evaluations were recorded: vital signs (HR and BP); 12-lead ECG; adverse events inquiry; concomitant treatment changes; and location of the patient (ER, ward, CCU/ICU), During the study drug infusion, continuous ECG monitoring was undertaken at least for 24 hours.

On re-administration Days 1 (at 24-48 hours), 2 (at 48-72 hours), and 3 (at 72-96 hours), the dobutamine/placebo infusion, if ongoing, may have been continued or may have been stopped gradually at any time at the investigator's discretion. In addition, the following evaluations were recorded: vital signs (HR and BP); adverse events inquiry; concomitant treatment changes; and location of the patient (ER, ward, CCU/ICU). On re-administration Day 2, a 12-lead ECG was performed. If the patient was discharged on re-administration Day 1, 2, or 3, then assessments for that day/visit were completed prior to discharge. The assessments for the subsequent visits were not required.

On re-administration Day 4 and consecutive days during the rehospitalization, the dobutamine/placebo infusion, if ongoing, may have been continued or may have been stopped gradually at any time at the investigator's discretion. In addition, the following evaluations were recorded: adverse events inquiry; concomitant treatment changes; and location of the patient (ER, ward, CCU/ICU).

On discharge from the rehospitalization (or death during the rehospitalization) the following were completed in the CRF: patient status (alive or dead, date, time, location and cause of death); date of discharge and to where discharged (home, other hospital—reason for transfer, nursing home—reason for transfer); and concomitant treatment at discharge.

Patients either returned to the hospital or were contacted by telephone on Day 31 (±5 days) following the respective re-administration. The following evaluations were recorded: patient status (alive or dead, date, time, location and cause of death) and adverse events inquiry.

### Criteria for Evaluation

### Efficacy:

Primary Efficacy Variable: “All-cause mortality” during the 180 days following randomization.

Secondary Efficacy Variables: All-cause mortality during the 31 days following randomization; mean change in plasma BNP concentration from baseline to 24 hours after start of the study drug infusion; number of days alive out of hospital (DAOH) during the 180 days following randomization; Patient's Assessment of Dyspnea at 24 hours following randomization; Patient's Global Assessment at 24 hours following randomization; and cardiovascular, mortality during the 180 days following randomization.

Additional efficacy variables: Following the completion of the study and prior to unblinding, the following additional ad hoc variables were identified and specified in the SAP. All-cause mortality during 90 days, cardiovascular mortality during 31 and 90 days, all-cause morbidity, DAOH during 31 and 90 days, study medication effects on renal function and in patients with renal impairment, concomitant IV medication, use of diuretics, IV vasodilators, and IV inotropes during the five days following initiation of study drug, decrease in BNP of at least 30% at 24, 72 and 120 hours, change from baseline in NT-proBNP concentration. Additional analyses for primary and secondary efficacy variables were also performed.

Following the completion of the study and prior to the unblinding, additional ad hoc variables were identified and pre-specified: After unblinding additional post hoc variables were identified and added.

### Pharmacokinetic: Not applicable.

### Safety: Heart rate (HR), blood pressure (BP), 12-lead ECG, laboratory tests (biochemistry and hematology), and adverse events.

### Statistical Methods

### Efficacy:

The primary analysis was to compare the all-cause mortality between the levosimendan and dobutamine randomized patients during 180 days following randomization for the ITT-population using a Cox-proportional-hazards model with treatment group as the effect. The hazard ratio between the treatments and its 95% confidence interval (hereinafter “CI”) and associated p-value were computed using a Cox-proportional-hazards model with treatment as a factor. Kaplan-Meier curves using the above event and censoring times were drawn by treatment group to visualize the effect of the treatments. To maintain the overall significance level of 0.05, the comparison was declared significant if the p-value from the Cox-proportional-hazards model for treatment effect reached a level of 0.047 or less at the final analysis, based on a total of 358 deaths and after considering two interim analyses at p-value of 0.005 or less using the Haybittle-Peto boundary. The primary efficacy variable was analyzed with stratification for previous CHF, acute MI at initial hospitalization and using the generalized Wilcoxon test to check the sensitivity of any treatment effect to the variation of baseline diseases and statistical methodology.

All secondary efficacy variables were evaluated using the ITT population. Following the completion of the study and prior to the unblinding, the hierarchical order of secondary efficacy variables was specified. The change in plasma BNP concentration from baseline to 24 hours was also added.

All-cause mortality during the 31 days following randomization was evaluated by the hazard ratio between the treatments using a Cox-proportional-hazards model with effects for treatment.

The change in plasma BNP concentration from baseline to 24 hours was analyzed using ANCOVA with main effect for treatment and baseline value as a covariate and the Kruskal-Wallis test.

Number of DAOH during the 180 days following randomization was compared between treatment groups by the CMH test of row mean table scores with treatment as effect.

The change in the Patient's Assessment of Dyspnea at 24 hours following randomization was analyzed by treatment group using the CMH test of row mean table scores.

The change in the Patient's Global Assessment at 24 hours following randomization was analyzed by treatment group in the same way as for the dyspnea assessment.

Cardiovascular mortality during 180 days following randomization was analyzed with methodology similar to the all-cause mortality during 31 days.

Following the completion of the study and prior to the unblinding, additional ad hoc variables were identified and pre-specified: After unblinding additional post hoc variables were identified and added.

### Pharmacokinetic: Not applicable

### Safety:

Safety and tolerability were analyzed using the safety population, defined as all patients who were randomized and received any study drug.

Adverse events occurring prior to the start of the study drug infusion were not included in the safety analyses unless the severity of the event changed after the start of the study drug infusion.

Adverse events were collected up to 31 days from the start of the study drug infusion, as well as 31 days following all re-administrations of blinded study drug. Between Day 31 and 180, only serious adverse events with at least possible causality (or not assessable) were recorded. Adverse events were summarized by the number and percentages of patients, severity, and by time of onset of the event. Adverse events were classified by system organ class and by preferred term according to the Medical Dictionary for Regulatory Activities (hereinafter “MedDRA”) coding system. The incidence rates of adverse events were compared between treatment groups using a Fisher's Exact test. The incidence rates of adverse events were also tabulated by baseline blood-pressure strata and by events of special interest and compared between treatment groups using Fisher's Exact test. Adverse drug reactions, (i.e., adverse events with related or unknown causality to study drug) were tabulated separately. Serious adverse events, events leading to permanent discontinuation of the study drug infusion, and events with a fatal outcome were also tabulated separately.

For the laboratory safety variables, descriptive statistics for each variable at each time point were summarized by treatment group. An ANCOVA model was used to compare differences between treatment groups, with the baseline value as the covariate and main effect for treatment. Shift tables were produced for out-of-normal and out-of-alert values.

For mean systolic and diastolic blood pressures, calculated mean arterial pressure, and heart rate, comparison between treatment groups was performed separately for each evaluation time points using analysis of covariance ANCOVA, with baseline value as a covariate and main effect for treatment for absolute values and the analysis of covariance ANCOVA with treatment effect for changes from baseline.

The number of patients with an episode of symptomatic hypotension following the initial study drug infusion and following all re-administrations, and the number of such episodes, was tabulated separately with percentages and compared between the groups using Fisher's Exact test. The time to first onset, duration, and severity of hypotensive events were also tabulated with descriptive statistics.

The number of patients with an episode of tachycardia was tabulated with percentages and compared between treatment groups using the Fisher's Exact test. The time from start of the study drug infusion to first tachycardic event, duration, and severity (the highest heart rate during the episode) were tabulated with descriptive statistics by treatment group.

From the ECG recordings, the rhythm, conduction, and morphology were tabulated with percentages by treatment groups and visits. Descriptive statistics of the standard ECG intervals, including QT, PR, QRS and RR were tabulated. The primary correction method for the QTc interval was calculated using Fridericia's correction (“QTcF”). QT, Bazett's correction (“QTcB”), and QTcF were tabulated with descriptive statistics both in absolute values at each timepoint and as changes (in ms) from baseline. The maximum change after study drug administration was initiated was also reported. Comparison between treatment groups was performed for each visit separately using ANCOVA with the baseline value as covariate. Analysis of covariance for repeated measures was performed for data with all visits to determine the overall effect of the treatment.

Categorical analyses of the QTc interval calculated using both Bazett's correction and Fridericia's correction (QTcB and QTcF, respectively) were also performed.

The linear regression of QT versus RR interval was calculated at each time point separately and also presented as a plot at each timepoint (at baseline, 24-hours, 3, 5 and 31 days).

All QTc analyses were repeated in patients by baseline blood pressure category (SBP<100 mmHg or DBP<60 mmHg, SBP≧100 mmHg and DBP≧60 mmHg).

### Summary/Conclusions

Efficacy Results:

No statistically significant difference was observed between the levosimendan and dobutamine treatment groups in all-cause mortality during the 180 days following the start of the study drug infusion (26.1% and 27.9%, respectively, HR=0.91, 95% CI: 0.74 to 1.13; p=0.401) in patients with acute HF. Similarly, no statistically significant differences were observed between the treatment groups in all-cause or cardiovascular mortality during the 31 days following the start of the study drug infusion, or in all-cause mortality during the 90 days following the start of the infusion.

There were statistically significantly greater mean reductions in plasma BNP and NT-proBNP concentrations from baseline to 24, 72, and 120 hours after the start of the study drug infusion in the levosimendan group compared to the dobutamine group (p<0.001 at all time points).

The mean number of DAOH during the 180 days following the start of study drug infusion was similar between the groups (120.2 vs. 116.6 days for levosimendan and dobutamine, respectively).

Patients in both the levosimendan and dobutamine treatment groups demonstrated improvements in both the Patient's Assessment of Dyspnea and the Patient's Global Assessment at 24 hours, with similar proportions being at least moderately improved. The differences between the treatment groups were not statistically significant.

All-cause mortality at 5, 14, 31, and 180 days after start of the study drug infusion was numerically lower in the levosimendan group compared to the dobutamine group. However, these treatment differences did not reach statistical significance.

Among patients who did not have a previous history of heart failure at baseline, all-cause mortality was numerically greater in the levosimendan group compared to the dobutamine group at 180 days.

Among patients who had received β-blockers at baseline, all-cause mortality was numerically lower in the levosimendan group compared to the dobutamine group at all time points. The difference between the levosimendan and dobutamine treatment groups was statistically significant at Day 5 (1% vs. 5%, p=0.014).

Pharmacokinetic Results. Not applicable.

### Safety Results:

The proportion of patients reporting at least one treatment-emergent adverse event was similar in the two treatment groups: 78% in the levosimendan group and 76% in the dobutamine group.

The most commonly (≧5.0% of patients in either treatment group) reported treatment-emergent events were hypotension (15%), cardiac failure (12%), atrial fibrillation and hypokalemia (9% each), ventricular tachycardia and headache (8% each), nausea (7%), ventricular extrasystoles and insomnia (6% each), and tachycardia and chest pain (5% each) in the levosimendan group; and cardiac failure (17%), hypotension (14%), ventricular tachycardia, nausea, and chest pain (7% each), atrial fibrillation and hypokalemia (6% each) and tachycardia and headache (5% each) in the dobutamine group.

A statistically significantly greater proportion of patients in the levosimendan group compared to the dobutamine group reported treatment-emergent atrial fibrillation (9% and 6%, p=0.048), hypokalemia (9% and 6%, p=0.022), headache (8% and 5%, p=0.010), and agitation (1% and 0%, p=0.015). Additionally, a statistically significantly higher proportion of patients in the dobutamine group compared to the levosimendan group reported treatment-emergent cardiac failure (17% and 12%, p=0.019).

With respect to treatment-related adverse events, there were significantly more cases of angina pectoris in the dobutamine group (0% and 1%, p=0.008). There were no statistical differences observed between treatment groups for the incidence of any other treatment-related adverse event.

The incidence of Torsades de Pointes was similar in the levosimendan and dobutamine treatment groups (1% each).

The most common treatment-emergent adverse-event categories of special interest (consisting of grouped MedDRA preferred terms) reported in both treatment groups were heart failure, ventricular arrhythmias, hypotension, and atrial arrhythmias. A statistically significantly greater proportion of patients in the levosimendan group compared to the dobutamine group experienced decreased potassium (10% and 7%, p=0.027).

Analysis of treatment-related adverse events of special interest (grouped terms) revealed that a statistically significantly greater proportion of patients in the dobutamine group compared to the levosimendan group experienced acute coronary syndromes/ischemia (<1% and 2%, p=0.011).

The proportion of patients who experienced adverse events with a fatal outcome was similar in the levosimendan and dobutamine groups (14% and 15%, respectively). Adverse events with a fatal outcome that were considered by the investigator to be related to study drug occurred in four levosimendan patients and two dobutamine patients. Each of the remaining adverse events with a fatal outcome was considered by the investigator to be not related to study drug.

The proportion of patients experiencing serious adverse events during the study was similar in the levosimendan and dobutamine treatment groups (30% and 33%, respectively).

Seventy-eight patients (33 levosimendan and 45 dobutamine) had at least one adverse event leading to permanent discontinuation of initial or subsequent (re-administration) study drug infusion.

There were no clinically relevant mean changes from baseline for any of the laboratory variables in either treatment group.

The proportion of patients with a symptomatic hypotensive episode during the initial treatment period was similar in both the levosimendan and dobutamine groups (9% and 7%).

The number of patients with tachycardia episodes meeting the protocol definition of ≧140 beats/min for at least 10 minutes during the initial treatment period was 48 in the levosimendan group and 52 in the dobutamine group.

### Conclusions:

General Conclusions:

The primary results revealed that there was no statistical difference between levosimendan and dobutamine in all-cause mortality at 180 days after start of study drug infusion in patients with acute HF, though levosimendan provided a numerical survival advantage through 31 days and at 180 days versus dobutamine in patients with acute HF. This benefit was more prominent in patients receiving β-blocker therapy at baseline and with a history of heart failure. These survival results are consistent with greater decreases in BNP observed with levosimendan treatment. The safety profile of the two agents is similar suggesting that levosimendan has a favorable benefit/risk profile.

Blood Pressure Conclusions:

The data in Tables 20-24 below demonstrate that patients having a baseline SBP<100 mm Hg or DBP<60 mmHg and received levosimendan or dobutamine were at a higher right of mortality at 5 days, 14 days, 31 days, 90 days and 180 days compared to patients having a baseline SBP≧100 mmHg or DBP≧60 mmHg.

TABLE 20
5 Days

| | | | | Mortality Rate |
|---|---|---|---|---|
| | | Mortality Rate | | for |
| | SBP < 100 mmHg | for SBP < 100 mmHg | SBP ≧ 100 mmHg | SBP ≧ 100 mmHg |
| | or DBP < 60 mmHg | or DBP < 60 mmHg | or DBP ≧ 60 mmHg | or DBP ≧ 60 mmHg |
| Levosimendan | 14/198 | 7.1 | 15/464 | 3.2 |
| Dobutamine | 14/182 | 7.7 | 25/478 | 5.2 |

TABLE 21
14 Days

| | | | | Mortality Rate |
|---|---|---|---|---|
| | | Mortality Rate | | for |
| | SBP < 100 mmHg | for SBP < 100 mmHg | SBP ≧ 100 mmHg | SBP ≧ 100 mmHg |
| | or DBP < 60 mmHg | or DBP < 60 mmHg | or DBP ≧ 60 mmHg | or DBP ≧ 60 mmHg |
| Levosimendan | 24/198 | 12.1 | 34/464 | 7.3 |
| Dobutamine | 28/182 | 15.4 | 40/478 | 8.4 |

TABLE 22
31 Days

| | | | | Mortality Rate |
|---|---|---|---|---|
| | | Mortality Rate | | for |
| | SBP < 100 mmHg | for SBP < 100 mmHg | SBP ≧ 100 mmHg | SBP ≧ 100 mmHg |
| | or DBP < 60 mmHg | or DBP < 60 mmHg | or DBP ≧ 60 mmHg | or DBP ≧ 60 mmHg |
| Levosimendan | 34/198 | 17.2 | 44/464 | 9.5 |
| Dobutamine | 37/182 | 20.3 | 53/478 | 11.1 |

TABLE 23
90 Days

| | | | | Mortality Rate |
|---|---|---|---|---|
| | | Mortality Rate | | for |
| | SBP < 100 mmHg | for SBP < 100 mmHg | SBP ≧ 100 mmHg | SBP ≧ 100 mmHg |
| | or DBP < 60 mmHg | or DBP < 60 mmHg | or DBP ≧ 60 mmHg | or DBP ≧ 60 mmHg |
| Levosimendan | 57/198 | 28.2 | 81/464 | 17.5 |
| Dobutamine | 58/182 | 31.9 | 78/478 | 16.3 |

TABLE 24
180 Days*

| | | | | Mortality Rate |
|---|---|---|---|---|
| | | Mortality Rate | | for |
| | SBP < 100 mmHg | for SBP < 100 mmHg | SBP ≧ 100 mmHg | SBP ≧ 100 mmHg |
| | or DBP < 60 mmHg | or DBP < 60 mmHg | or DBP ≧ 60 mmHg | or DBP ≧ 60 mmHg |
| Levosimendan | 71/198 | 35.9 | 101/464 | 21.8 |
| Dobutamine | 73/182 | 40.1 | 110/478 | 23.0 |

*Note for Tables 20-24, relative risks were intra-group comparisons. For example the relative risk of 2.055 at Day 5 represents an intra-group comparison of levosimendan patients with a baseline SBP < 100 mmHg to levosimendan patients that did not have baseline SPB < 100 mmHg; 7.4 divided by 3.6 = 2.055.

One skilled in the art would readily appreciate that the present invention is well adapted to carry out the objects and obtain the ends and advantages mentioned, as well as those inherent therein. The compositions, formulations, methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features modification and variation of the concepts herein disclosed may be resorted to by those skilled in the art, and that such modifications and variations are considered to be within the scope of this invention as defined by the appended claims.

What is claimed is:

1. A method of predicting an improved clinical outcome in a patient diagnosed with congestive heart failure, the method comprising the steps of:
(a) obtaining a systolic blood pressure, diastolic blood pressure or mean arterial pressure from a patient;
(b) determining whether the systolic blood pressure, diastolic blood pressure or mean arterial pressure of said patient fits at least one predetermined criterion showing a likelihood of an improved clinical outcome; and
(e) predicting said improved clinical outcome based upon the determination in step (b).

2. The method of claim 1 wherein the predetermined criterion showing a likelihood of improved clinical outcome is a systolic blood pressure less than, equal to or greater, than about 100 mmHg.

3. The method of claim 1 wherein the predetermined criterion showing a likelihood of improved clinical outcome is a diastolic blood pressure less than, equal to or greater than about 60 mmHg.

4. The method of claim 1 wherein the predetermined criterion showing a likelihood of improved clinical outcome is a mean arterial pressure less than, equal to or greater than about 75 mmHg.

5. The method of claim 1 wherein the patient is suffering from acutely decompensated congestive heart failure.

6. The method of claim 1 wherein a systolic blood pressure equal to or greater than about 100 mmHg indicates an increased likelihood of an improved clinical outcome.

7. The method of claim 1 wherein a systolic blood pressure less than about 100 mmHg indicates a decreased likelihood of an improved clinical outcome.

8. A method for determining whether or not to begin a course of monotherapy with an inotropic drug or a vasodilator in a patient diagnosed with congestive heart failure, the method comprising the steps of:
(a) obtaining a systolic blood pressure, diastolic blood pressure or mean arterial pressure from a patient;
(b) analyzing the systolic blood pressure, diastolic blood pressure or mean arterial pressure of said patient to determine whether said blood pressure, diastolic blood pressure or mean diastolic blood pressure fits at least one predetermined criterion; and
(c) determining whether to begin a course of monotherapy in the patient based upon the analysis in step (b).

9. The method of claim 8 wherein the predetermined criterion is a systolic blood pressure less than, equal to or greater than about 100 mmHg.

10. The method of claim 8 wherein the predetermined criterion is a diastolic blood pressure less than, equal to or greater than about 60 mmHg.

11. The method of claim 8 wherein the predetermined criterion is a mean arterial pressure less than, equal to or greater than about 75 mmHg.

12. The method of claim 8 wherein in step (c), a determination is made not to begin a course of monotherapy with an inotropic drug or a vasodilator because the patient was determined to have a systolic blood pressure less than about 100 mmHg in step (b).

13. The method of claim 8 wherein in step (c), a determination is made to begin a course of monotherapy with an inotropic drug or a vasodilator because the patient was determined to have a systolic blood pressure about equal to or greater than about 100 mmHg in step (b).

14. The method of claims 8 or 13 further comprising the step of administering an effective amount of an inotropic drug or a vasodilator to the patient based upon the determination in step (c).

15. The method of claim 14 wherein the inotropic drug or vasodilator is administered to the patient orally, buccally, intravenously, intramuscularly, subcutaneously or by inhalation.

16. The method of claim 14 wherein the inotropic drug is levosimendan, dopamine, dobutamine, inamrinone, milrinone, dopexamine or digoxin.

17. The method of claim 14 wherein the vasodilator is hydralazine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate or isosorbide mononitrate.

18. The method of claim 8 wherein the patient is suffering from acutely decompensated congestive heart failure.

19. A method for determining whether or not to begin a course of combination therapy with at least one inotropic drug, at least one vasodilator or combinations thereof in a patient diagnosed with congestive heart failure, the method comprising the steps of:
(a) obtaining a systolic blood pressure, diastolic blood pressure or mean arterial pressure from a patient;
(b) analyzing the systolic blood pressure, diastolic blood pressure or mean arterial pressure of said patient to determine whether said blood pressure, diastolic blood pressure or mean diastolic blood pressure fits at least one predetermined criterion; and
(c) determining whether to begin a course of combination therapy with at least one inotropic drug, at least one vasodilator or combinations thereof in the patient based upon the analysis in step (b).

20. The method of claim 19 wherein the predetermined criterion is a systolic blood pressure less than, equal to or greater than about 100 mmHg.

21. The method of claim 19 wherein the predetermined criterion is a diastolic blood pressure less than, equal to or greater than about 60 mmHg.

22. The method of claim 19 wherein the predetermined criterion is a mean arterial pressure less than, equal to or greater than about 75 mmHg.

23. The method of claim 19 wherein in step (c), a determination is made not to begin a course of combination therapy with at least one inotropic drug, at least one vasodilator or combinations thereof because the patient was determined to have a systolic blood pressure less than about 100 mmHg in step (b).

24. The method of claim 19 wherein in step (c), a determination is made to begin a course of combination therapy with at least one inotropic drug, at least one a vasodilator or combinations thereof because the patient was determined to have a systolic blood pressure equal to or greater than about 100 mmHg in step (b).

25. The method of claims 19 or 24 further comprising the step of administering an effective amount at least one inotropic drug, at least one vasodilator or combinations thereof to the patient based upon the determination in step (c).

26. The method of claim 25 wherein the at least one inotropic drug, at least one vasodilator or combinations thereof is administered to the patient orally, buccally, intravenously, intramuscularly, subcutaneously or by inhalation.

27. The method of claim 25 wherein the inotropic drug is levosimendan, dopamine, dobutamine, inamrinone, milrinone, dopexamine or digoxin.

28. The method of claim 25 wherein the vasodilator is hydralazine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate or isosorbide mononitrate.

29. The method of claim 19 wherein the patient is suffering from acutely decompensated congestive heart failure.

30. A method for determining whether to continue or discontinue a course of monotherapy in a patient diagnosed with congestive heart failure wherein said patient is currently being treated with an inotropic drug or a vasodilator, the method comprising the steps of:
(a) obtaining a systolic blood pressure, diastolic blood pressure or mean arterial pressure from a patient;
(b) analyzing the systolic blood pressure, diastolic blood pressure or mean arterial pressure of said patient to determine whether said systolic blood pressure, diastolic blood pressure or mean diastolic blood pressure fits at least one predetermined criterion; and
(c) determining whether to continue or discontinue the course of monotherapy with the inotropic drug or vasodilator in said patient based upon the analysis in step (b).

31. The method of claim 30 wherein the predetermined criterion is a systolic blood pressure less than, equal to or greater than about 100 mmHg.

32. The method of claim 30 wherein the predetermined criterion is a diastolic blood pressure less than, equal to or greater than about 60 mmHg.

33. The method of claim 30 wherein the predetermined criterion is a mean arterial pressure less than, equal to or greater than about 75 mmHg.

34. The method of claim 30 wherein in step (c), a determination is made not to continue a course of monotherapy because the patient was determined to have a systolic blood pressure less than about 100 mmHg in step (b).

35. The method of claim 30 wherein in step (c), a determination is made to continue a course of monotherapy because the patient was determined to have a systolic blood pressure equal to or greater than about 100 mmHg in step (b).

36. The method of claim 30 wherein the inotropic drug is levosimendan, dopamine, dobutamine, inamrinone, milrinone, dopexamine or digoxin.

37. The method of claim 30 wherein the vasodilator is hydralazine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate or isosorbide mononitrate.

38. The method of claim 30 wherein the patient is suffering from acutely decompensated congestive heart failure.

39. A method for determining whether to continue or discontinue a course of combination therapy in a patient diagnosed with congestive heart failure wherein said patient is currently being treated with a combination of at least one inotropic drug, at least one vasodilator or combinations thereof the method comprising the steps of:
(a) obtaining a systolic blood pressure, diastolic blood pressure or a mean arterial pressure from a patient;
(b) analyzing the systolic blood pressure, diastolic blood pressure or mean arterial pressure of said patient to determine whether said systolic blood pressure, diastolic blood pressure or mean diastolic blood pressure fits at least one predetermined criterion; and
(c) determining whether to continue or discontinue the course of combination therapy with the at least one inotropic drug, at least one vasodilator or combinations thereof in said patient based upon the analysis in step (b).

40. The method of claim 39 wherein the predetermined criterion is a systolic blood pressure less than, equal to or greater than about 100 mmHg.

41. The method of claim 39 wherein the predetermined criterion is a diastolic blood pressure less than, equal to or greater than about 60 mmHg.

42. The method of claim 39 wherein the predetermined criterion is a mean arterial pressure less than, equal to or greater than about 75 mmHg.

43. The method of claim 39 wherein in step (c), a determination is made not to continue a course of combination therapy because the patient was determined to have a systolic blood pressure less than about 100 nm mg in step (b).

44. The method of claim 39 wherein in step (c), a determination is made to continue a course of combination therapy because the patient was determined to have a systolic blood pressure equal to or greater than about 100 mmHg in step (b).

45. The method of claim 40 wherein the inotropic drug is levosimendan, dopamine, dobutamine, inamrinone, milrinone, dopexamine or digoxin.

46. The method of claim 40 wherein the vasodilator is hydralazine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate or isosorbide mononitrate.

47. The method of claim 40 wherein the patient is suffering from acutely decompensated congestive heart failure.

48. A method of optimizing an existing drug treatment regimen in a patient diagnosed with congestive heart failure and receiving at least one inotropic drug, at least one vasodilator, at least one beta-blocker or combinations thereof, the method comprising the steps of:
(a) obtaining a systolic blood pressure, diastolic blood pressure or mean arterial pressure from a patient;
(b) analyzing the systolic blood pressure, diastolic blood pressure or mean arterial pressure of said patient to determine whether said systolic blood pressure, diastolic blood pressure or mean diastolic blood pressure fits at least one predetermined criterion; and
(c) optimizing the treatment regimen in the patient based upon the analysis in step (b).

49. The method of claim 48 wherein the predetermined criterion is a systolic blood pressure less than, equal to or greater than about 100 mmHg.

50. The method of claim 48 wherein the predetermined criterion is a diastolic blood pressure less than, equal to or greater than about 60 mmHg.

51. The method of claim 48 wherein the predetermined criterion is a mean arterial pressure less than, equal to or greater than about 75 mmHg.

52. The method of claim 48 wherein in step (c), a determination is made to optimize the treatment regimen by discontinuing the treatment of the patient with at least one inotropic drug, at least one vasodilator, at least one beta-blocker or combinations thereof based upon the determination in step (b) that the patient has a systolic blood pressure less than about 100 mmHg.

53. The method of claim 48 wherein in step (c), the treatment regimen is optimized by substituting an effective amount of at least one different inotropic drug, at least one different vasodilator, at least one different beta-blocker or different combinations thereof that are not currently being used in the existing treatment regimen for at least one inotropic drug, at least one vasodilator, at least one beta-blocker or combinations thereof that is currently being used in the existing treatment regimen based upon the determination in step (b) that the patient has a systolic blood pressure about equal to or greater than about 100 mmHg.

54. The method of claim 48 wherein in step (c), the treatment regimen is optimized by adding an effective amount of at least one inotropic drug, at least one vasodilator, at least one beta-blocker or combinations thereof that are not currently being used in the existing treatment regimen based upon the determination in step (b) that the patient has a systolic blood pressure about equal to or greater than about 100 mmHg.

55. The method of claims 52, 53 or 54 wherein the inotropic drug is levosimendan, dopamine, dobutamine, inamrinone, milrinone, dopexamine or digoxin.

56. The method of claims 52, 53 or 54 wherein the vasodilator is hydralazine, hydralazine hydrochloride, nicorandil, fenoldopam, natriuretic peptides, natrecor; nesiritide, nitroprusside, nitroprusside sodium, nipride, milrinone, primacor, nitroglycerin, glyceryl trinitrate, isosorbide dinitrate or isosorbide mononitrate.

57. The methods of claims 52, 53 or 54 wherein the beta-blocker is labetalol, carvedilol, atenolol, esmolol, esmolol hydrochloride, metoprolol, metoprolol succinate, metoprolol tartrate, bisoprolol fumarate, bisoprolol, propranolol or propranolol hydrochloride.

58. The method of claim 48 wherein the patient is suffering from acutely decompensated congestive heart failure.

59. A pharmaceutical drug product comprising:
(a) a pharmaceutical drug for the treatment of congestive heart failure in a pharmaceutically-acceptable form for administration to a human, and
(b) printed media or labeling accompanying said pharmaceutical drug in said pharmaceutically acceptable form, wherein said printed media or labeling discloses the method as in any one of claims 1, 8, 19, 30, 39 or 48.

60. A pharmaceutical drug product comprising:
(a) levosimendan in a pharmaceutically-acceptable form for administration to a human, and
(b) printed media or labeling accompanying said levosimendan, wherein said printed media or labeling discloses the method as in any one of claims 1, 8, 19, 30 39 or 48.

61. A business method comprising, providing a drug for the treatment of congestive heart failure in a pharmaceutically-acceptable form for administration to a human, and
encouraging a decision maker to refer to printed or electronic media disclosing a method as in any one of claims 1, 8, 19, 30, 39 or 48.

62. A business method comprising, providing levosimendan in a pharmaceutically-acceptable form for administration to a human, and encouraging a decision maker to refer to printed or electronic media disclosing a method as in any one of claims 1, 8, 19, 30, 39 or 48.

63. A method of promoting the sale or use of a drug for the treatment of congestive heart failure, the method comprising:
(a) providing said drug in a pharmaceutically-acceptable form for administration to a human, and
(b) providing a customer with printed, graphic, or electronic media disclosing a method as in any one of claims 1, 8, 19, 30, 39 or 48.

64. A method of promoting the sale or use of levosimendan, the method comprising:
(a) providing said levosimendan in a pharmaceutically-acceptable form for administration to a human, and
(b) providing a customer with printed, graphic, or electronic media disclosing a method as in any one of claims 1, 8, 19, 30, 39 or 48.
